(19) United States
(12) Patent Application Publication
(10) Pub. No.: US 2025/0109429 A1
(43) Pub. Date: Apr. 3, 2025

(54) SELF ASSEMBLY OF BEADS ON SUBSTRATES

(71) Applicant: Ultima Genomics, Inc., Fremont, CA (US)

(72) Inventors: Aklilu WORKU, San Francisco, CA (US); Robert ONO, Palo Alto, CA (US); Daniel MAZUR, San Diego, CA (US); Gilad ALMOGY, Palo Alto, CA (US)

(21) Appl. No.: 18/919,271

(22) Filed: Oct. 17, 2024

Related U.S. Application Data
(63) Continuation of application No. PCT/US2023/019297, filed on Apr. 20, 2023.
(60) Provisional application No. 63/333,311, filed on Apr. 21, 2022.

(51) Int. Cl. C12Q 1/6806; C01B 33/113; C12Q 1/6869

(52) U.S. Cl. CPC C12Q 1/6806; C01B 33/113; C12Q 1/6869

(57) ABSTRACT

Provided herein are methods, systems, and kits for generating a self-assembled monolayer of beads adjacent to (e.g., on or across) a substrate. The methods, systems, and kits provided herein may comprise contacting a substrate with a plurality of beads, which may comprise nucleic acid molecules coupled thereto, and providing conditions sufficient for the plurality of beads to self-assemble adjacent to the substrate. Sequencing of the nucleic acid molecules may be performed.

### CROSS REFERENCE

This application is a continuation of International Patent Application No. PCT/US2023/019297, filed Apr. 20, 2023, which claims the benefit of U.S. Provisional Patent Application No. 63/333,311, filed Apr. 21, 2022, which is entirely incorporated herein by reference in its entirety.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious efforts. For example, spatial heterogeneity of samples presented on a substrate for imaging may result in waste of reagents and unutilized substrate space.

### SUMMARY

Recognized herein is a need for facile methods of loading samples coupled to particles (e.g., beads) on to substrates in a highly dense fashion for further characterization or analysis (e.g., via nucleic acid sequencing).

In an aspect, disclosed herein is a method for self-assembly of a plurality of beads, comprising: (a) providing a substrate and the plurality of beads, wherein the substrate is unpatterned and substantially planar; (b) dispensing the plurality of beads adjacent to the substrate; and (c) subjecting the substrate or the plurality of beads to conditions sufficient for self-assembly of the plurality of beads adjacent to the substrate.

In some embodiments, the substrate is a solid or semi-solid substrate. In some embodiments, the plurality of beads is a plurality of solid or semi-solid beads. In some embodiments, the plurality of beads is formed from a metal, a ceramic, a polymer, or glass, or a combination thereof. In some embodiments, the polymer is a gel. In some embodiments, the gel is a hydrogel. In some embodiments, the plurality of beads is electrostatically charged. In some embodiments, the plurality of beads is negatively charged, and wherein the substrate or portion thereof is positively charged. In some embodiments, the portion thereof is a surface of the substrate. In some embodiments, the plurality of beads comprises a plurality of nucleic acid molecules coupled thereto. In some embodiments, the plurality of nucleic acid molecules comprises a deoxyribonucleic acid (DNA) molecule. In some embodiments, the DNA molecule is double-stranded. In some embodiments, the DNA molecule is single-stranded. In some embodiments, in (a), the plurality of beads is provided in a solution, wherein the solution comprises single-stranded binding proteins. In some embodiments, the method further comprises sequencing the plurality of nucleic acid molecules. In some embodiments, the sequencing comprises flow sequencing, which flow sequencing comprises (i) providing a reagent comprising a first plurality of nucleotides to the plurality of beads or the substrate and (ii) detecting a nucleotide from the first plurality of nucleotides. In some embodiments, the flow sequencing further comprises (iii) providing an additional reagent comprising a second plurality of nucleotides to the plurality of beads or the substrate and (iv) detecting an additional nucleotide from the second plurality of nucleotides. In some embodiments, the first plurality and the second plurality of nucleotides are of a same nucleotide base type. In some embodiments, the first plurality and the second plurality of nucleotides are of different nucleotide base types. In some embodiments, beads of the plurality of beads are from about 0.1 microns to about 10 microns in diameter. In some embodiments, the method further comprises, prior to (b), wetting the substrate. In some embodiments, the wetting comprises wetting the substrate with an ionic buffer. In some embodiments, the ionic buffer comprises magnesium. In some embodiments, the ionic buffer comprises magnesium chloride. In some embodiments, the magnesium chloride is provided at a molarity of from about 10 to about 50 millimolar (mM). In some embodiments, the wetting renders the substrate hydrophilic. In some embodiments, the method further comprises treating the substrate prior to (b). In some embodiments, the treating comprises depositing a silane adjacent to the substrate. In some embodiments, the silane is an amino silane. In some embodiments, the amino silane is 3-aminopropyltrimethoxysilane (APTMS). In some embodiments, the silane is deposited on the substrate using vapor deposition. In some embodiments, the substrate comprises a silicon wafer. In some embodiments, the substrate comprises a silicon oxide layer. In some embodiments, the substrate comprises a glass wafer. In some embodiments, the glass wafer comprises a liquid crystal display. In some embodiments, the substrate does not comprise topographical features. In some embodiments, (a) comprises providing the plurality of beads in a solution, and wherein (b) comprises contacting the substrate with the solution. In some embodiments, (b) comprises providing the solution in one or more droplets to the substrate and allowing the solution to spread adjacent to the substrate. In some embodiments, the allowing comprises incubating the solution on the substrate. In some embodiments, the incubating is performed for about 20 to about 120 minutes. In some embodiments, the method further comprises, rotating the substrate to disperse the plurality of beads across the substrate. In some embodiments, the rotating is performed from about 500 revolutions per minute (rpm) to about 8000 revolutions per minute (rpm). In some embodiments, the method further comprises drying the substrate subsequent to the rotating. In some embodiments, the drying is performed for about 20 minutes. In some embodiments, the drying is performed at ambient temperature. In some embodiments, the drying is performed at a temperature from about 25 degrees Celsius to about 200 degrees Celsius. In some embodiments, (b) comprises translating the substrate relative to the solution, thereby contacting the substrate with the plurality of beads. In some embodiments, the contacting is performed for from about 1 minute to about 60 minutes. In some embodiments, the contacting is performed for about 60 minutes. In some embodiments, the contacting is performed for about 120 minutes. In some embodiments, the method further comprises, drying the substrate subsequent to the contacting. In some embodiments, the drying is performed for from about 1 minute to about 60 minutes. In some embodiments, the drying is performed for 15 minutes. In some embodiments, the drying is performed for 30 minutes. In some embodiments, subsequent to (c), the plurality of beads is arranged in a self-assembled monolayer. In some embodiments, self-assembled monolayer is substantially uniform. In some embodiments, the self-assembled monolayer has a bead-to-bead tolerance of approximately twice the diameter of a bead of the plurality of beads. In some embodiments, the self-assembled monolayer is arranged such that individual beads of the plurality of beads are distinguishable by microscopy. In some embodiments, the method further comprises subjecting the substrate to conditions sufficient to decrease the size of the plurality of beads. In some embodiments, the conditions sufficient to decrease the size of the plurality of beads comprises applying a shrinking buffer to the plurality of beads. In some embodiments, the shrinking buffer comprises polyethylene glycol (PEG). In some embodiments, the PEG comprises PEG 4000 or PEG 8000. In some embodiments, the PEG is provided at a 10% w/v concentration. In some embodiments, the shrinking buffer comprises magnesium salts. In some embodiments, the magnesium salts comprise magnesium chloride. In some embodiments, the magnesium chloride is provided at a 50 mM concentration. In some embodiments, the shrinking buffer comprises spermine. In some embodiments, the spermine is provided at a molarity from about 5 mM to about 50 mM.

In another aspect, disclosed herein is a kit, comprising: a substrate, wherein the substrate is unpatterned and substantially planar; a plurality of beads; and instructions for forming a self-assembled monolayer of the plurality of beads adjacent to the substrate.

In some embodiments, the substrate is a solid or semi-solid substrate. In some embodiments, the plurality of beads is a plurality of solid or semi-solid beads. In some embodiments, the plurality of beads comprises a plurality of nucleic acid molecules coupled thereto. In some embodiments, the plurality of nucleic acid molecules comprises a deoxyribonucleic acid (DNA) molecule. In some embodiments, the DNA molecule is double-stranded. In some embodiments, the kit further comprises a pre-wetting buffer. In some embodiments, the pre-wetting buffer comprises an ionic buffer. In some embodiments, the ionic buffer comprises magnesium. In some embodiments, the kit further comprises a shrinking buffer. In some embodiments, the shrinking buffer comprises polyethylene glycol (PEG). In some embodiments, the shrinking buffer comprises magnesium salts. In some embodiments, the magnesium salts comprise magnesium chloride.

In yet another aspect of the present disclosure, provided herein is a system, comprising: a substrate, wherein the substrate is unpatterned and substantially planar; and a plurality of beads, wherein: at least a first subset of the plurality of beads is in a substantially close-packed configuration, and at least a second subset of the plurality of beads is in a substantially monolayer configuration.

In some embodiments, the substrate is a solid or semi-solid substrate. In some embodiments, the plurality of beads is a plurality of solid or semi-solid beads. In some embodiments, the close-packed configuration comprises a center-to-center distance between neighboring beads of from 1 μm to 1.8 μm. In some embodiments, the plurality of beads comprises a plurality of nucleic acid molecules coupled thereto. In some embodiments, the plurality of nucleic acid molecules comprises a deoxyribonucleic acid (DNA) molecule. In some embodiments, the DNA molecule is double-stranded. In some embodiments, the first subset and the second subset are the same or substantially the same.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIG. 3 shows an example coating of a substrate with a hexagonal lattice of beads, as described herein.

FIG. 4 illustrates example systems and methods for loading a sample or a reagent onto a substrate, as described herein.

FIGS. 5A-5B illustrate multiplexed stations in a sequencing system.

FIG. 6 illustrates a computer system that is programmed or otherwise configured to implement methods provided herein.

FIG. 7 illustrates a process by which organosilane-functionalized surfaces may be generated.

FIGS. 8A-8C schematically show example workflows for self-assembly of beads on a substrate, in accordance with some embodiments. FIG. 8A schematically shows drop-coating a plurality of beads adjacent to a substrate. FIG. 8B schematically shows a dip-coating approach to providing a plurality of beads adjacent to a substrate. FIG. 8C schematically shows a spin-coating approach to providing a plurality of beads to a substrate.

FIG. 9 schematically shows a workflow for decreasing the size of a plurality of beads assembled adjacent to a substrate.

FIGS. 10A-10C show example data of a dip-coating method for providing a plurality of beads adjacent to a substrate, in accordance with some embodiments.

FIGS. 11A-11D show example data of a drop-coating method for providing a plurality of beads adjacent to a substrate, in accordance with some embodiments.

FIGS. 12A and 12B show example data of a spin-coating method for providing a plurality of beads adjacent to a substrate, in accordance with some embodiments.

FIGS. 13A-13E show representative images of bead aggregation on substrates (wafers) with a pitch size of 1.8 μm. The wafers were incubated in a buffer with different levels of Mg²⁺ (prewet) and the beads were incubated in a buffer with different levels of Mg²⁺ (load) before the beads were loaded onto the wafers. Circles highlight selected bead aggregates that were detected.

FIGS. 14A and 14B show representative images of bead aggregation on wafers with a pitch size of 1.5 μm. The wafers were incubated in a buffer with different levels of Mg²⁺ (prewet) and the beads were incubated in a buffer with different levels of Mg²⁺ (load) before the beads were loaded onto the wafers. Circles highlight example bead aggregates.

FIGS. 15A-15C show exemplary images of a substrate loaded with particles (e.g., beads). The images depict beads in a control imaging buffer (FIG. 15A), beads in an imaging buffer that includes 10% PEG-4000 (FIG. 15B), and beads in another imaging buffer that includes both 10% PEG-4000 and 50 mM MgCl₂ (FIG. 15C).

FIGS. 16A-16C show exemplary images of a substrate loaded with particles (e.g., beads). The images depict beads in the control imaging buffer (FIG. 16A), beads in an imaging buffer that includes 10% PEG-8000 (FIG. 16B), and beads in another imaging buffer that includes 10% PEG-8000 and 50 mM MgCl₂ (FIG. 16C).

FIG. 17 shows the effect of PEG-4000 on bead size. The graph illustrates exemplary measurements of bead diameter for beads loaded on a substrate and imaged in the presence of imaging buffers, where each buffer includes a titrated percentage of PEG-4000 (the x-axis). Each buffer further includes 50 mM of MgCl₂. The diameter of the beads was determined as relative full width at half maximum (FWHM) of the beads (the y-axis), where bead diameters were normalized to beads in an imaging buffer with no PEG-4000.

FIG. 18 shows the effect of MgCl₂ on bead size. The graph illustrates exemplary measurements of bead diameter (represented as FWHM of the beads (y-axis)) for beads loaded on a substrate and imaged in the presence of imaging buffers, where each buffer includes a titrated amount of MgCl₂ (the x-axis). Each buffer further includes 5% PEG-4000.

FIGS. 19A-19D show exemplary images of a substrate loaded with particles (e.g., beads). The images depict bead-loaded substrates that were prepared, prior to loading, with a prewetting buffer that lacks magnesium chloride (FIGS. 19A-19B) or comprises magnesium chloride (FIGS. 19C-19D). The substrates were incubated at room temperature after loading either for 60 minutes (FIGS. 19A and 19C) or for 75 minutes (FIGS. 19B and 19D).

FIGS. 20A-20D show exemplary images of substrate loaded with particles (e.g., beads), where the substrates have been prepared prior to loading with prewetting buffers having different concentrations of magnesium chloride, e.g., 50 mM MgCl₂, 100 mM MgCl₂, 150 mM MgCl₂, and 200 mM MgCl₂, respectively.

FIG. 21 shows the effect of bead concentration in loading. The graph illustrates exemplary percentage occupancy (e.g., how many possible locations on the substrate were occupied after loading) and exemplary efficiency (e.g., how many of the starting beads were successfully loaded onto the substrate). Both measurements were determined for positive, nucleic acid coupled beads.

FIGS. 22A-22D show exemplary images of a substrate loaded with particles (e.g., beads) at different overloading levels, as determined based on the number of available locations and the number of beads added, e.g., 1×, 1.2×, 1.4×, and 1.7× overloaded, respectively.

FIGS. 23A and 23B show exemplary images of a substrate loaded with particles (e.g., beads). The images depict bead-loaded substrates, where the substrates had been prepared prior to loading with prewetting buffer comprising 50M magnesium. The buffer used to load the beads comprised either 50M magnesium chloride and 1% w/v PEG (FIG. 23B) or no magnesium chloride and no PEG (FIG. 23A).

FIGS. 24A and 24B illustrate workflows utilizing click chemistry in bead-substrate associations. In FIG. 24A bead-bound circularized templates are deposited on a substrate via click chemistry. In FIG. 24B click chemistry bead-bound concatemers are deposited on a substrate.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-I antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxy acetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxy acetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:
(e.g., [A T G C A T G C A T G A T G A T G A T G C A T G C]).
   Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order and can be expressed in units of flow cycles. For example, where [A T G C] is identified as a 1^{st} flow cycle, and [A T G] is identified as a 2^{nd} flow cycle, the flow order of [A T G C A T G C A T GA T G A T G A T G C A T G C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle, 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity, and as such sequences would generally be considered “identical.”

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD), Hybrid TDI, complementary metal oxide semiconductor (CMOS) pseudo TDI device, or TDI line-scan camera.

The terms “dispense” and “disperse” may be used interchangeably herein. In some cases, dispensing may comprise dispersing and/or dispersing may comprise dispensing. Dispensing generally refers to distributing, depositing, providing, or supplying a reagent, solution, or other object, etc. Dispensing may comprise dispersing, which may generally refer to spreading.

The term “self-assembly,” as used herein, may generally refer to an organization of molecules or particles into ordered structures. The self-assembly may occur spontaneously or with the provision of a force or energy. The self-assembly may be reversible. The ordered structures may be joined via non-covalent interactions, e.g., van der Waals forces, ionic interactions, hydrophobic interactions, hydrogen bonding, etc. The self-assembly of particles (e.g., beads) may involve evaporation or capillary force. For example, a plurality of particles (e.g., beads) may be provided in a suspension comprising a liquid on a substrate, and the liquid may evaporate. Alternatively or in addition, a capillary force may help drive self-assembly of neighboring particles. Self-assembly may occur between the same types of particles or different types of particles (e.g., particles of varying size, compositions, charges, mechanical properties, etc.). The self-assembly may, in some instances, result in a substantially uniform layer (e.g., a monolayer) of the particles. The uniformity of the self-assembled particles may be measured, for example, by measuring the particle-to-particle distance and optionally, comparing particle-to-particle distance to a diameter of the particles. In some instances, the self-assembled particles may form a close-packed configuration (e.g., hexagonal, close-packed configuration).

### Sample Processing Methods

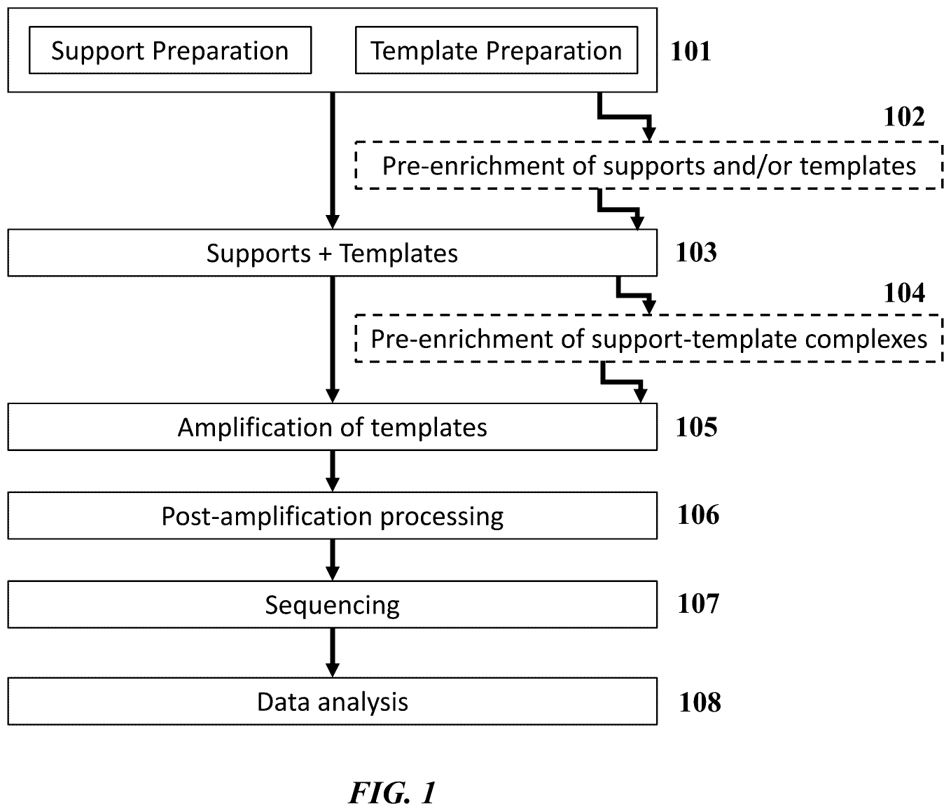

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be provided and/or prepared (101) to be compatible with downstream sequencing operations (e.g., 107). A support (e.g., bead) may help immobilize a template nucleic acid to a substrate, such as when the template nucleic acid is coupled to the support, and the support is in turn immobilized to the substrate. The support may further function as a binding entity to retain derivatives molecules (e.g., amplification products) from a same template nucleic acid together for downstream processing, such as for sequencing operations. This may be useful in distinguishing a colony from other colonies (e.g., on other supports) and generating amplified sequencing signals corresponding to a template nucleic acid. A support may comprise an oligonucleotide comprising one or more functional nucleic acid sequences. The oligonucleotide may be single-stranded, double-stranded, or partially double-stranded. For example, the oligonucleotide may comprise a capture sequence, a primer sequence, a sequencing primer sequence, a barcode sequence, a sample index sequence, a unique molecular identifier (UMI), a flow cell adapter sequence, an adapter sequence, a target sequence, a random sequence, a binding sequence (e.g., for a splint, primer, template nucleic acid, capture sequence, etc.), or any other functional sequence useful for a downstream operation, a complement thereof, or any combination thereof. The capture sequence may be configured to hybridize to a sequence of a template nucleic acid or derivative thereof. The support may comprise a plurality of oligonucleotides, for example on the order of 10, 10², 10³, 10⁴, 10⁵, 10⁶, 10⁷, or more molecules. The support may comprise a single species of oligonucleotide which comprise identical sequences. The support may comprise multiple species of oligonucleotides which have varying sequences. In some cases, the support comprises a single species of a primer (e.g., forward primer) for amplification. In some cases, the support comprises two species of primer (e.g., forward primer, reverse primer) for amplification. Devices, systems, methods, compositions, and kits for preparing and using support species are described in further detail in U.S. Patent Pub. No. 20220042072A1 and International Patent Pub. No. WO2022040557A2, each of which is entirely incorporated herein by reference for all purposes.

A support may comprise one or more capture entities, where a capture entity is configured for capture by a capturing entity. A capture entity may be coupled to or be part of an oligonucleotide coupled to the support. A capture entity may be coupled to or be part of the support. Examples of capture entity-capturing entity pairs and capturing entity-capture entity pairs include: streptavidin (SA)-biotin; complementary sequences; magnetic particle-magnetic field system; charged particle-electric field system; azide-cyclooctyne; thiol-maleimide; click chemistry pairs; cross-linking pairs; etc. (see Example 5 for click chemistry capture). The capture entity-capturing entity pair may comprise one or more chemically modified bases. A capture entity and capturing entity may bind, couple, hybridize, or otherwise associate with each other. The association may comprise formation of a covalent bond, non-covalent bond, releasable bond (e.g., cleavable bond that is cleavable upon application of a stimulus), and/or no bond. The capture entity may be capable of linking to a nucleotide. In some instances, the capturing entity may comprise a secondary capture entity, for example, for subsequent capture by a secondary capturing entity. The secondary capture entity and secondary capturing entity may comprise any one or more of the capturing mechanisms described elsewhere herein.

A support may comprise one or more cleavable moieties, also referred to herein as excisable moieties. The cleavable moiety may be coupled to or be part of an oligonucleotide coupled to the support. The cleavable moiety may be coupled to the support. A cleavable moiety may comprise any useful moiety that can be used to cleave an oligonucleotide (or portion thereof) from the support, or otherwise release a nucleic acid strand from the support and/or the oligonucleotide. A cleavable moiety may comprise a uracil, a ribonucleotide, methylated nucleotide, or other modified nucleotide that is excisable or cleavable using an enzyme (e.g., UDG, RNAse, APE1, MspJI, endonuclease, exonuclease, etc.). The cleavable moiety may comprise an abasic site or an analog of an abasic site (e.g., dSpacer), a dideoxyribose, a spacer, e.g., C3 spacer, hexanediol, triethylene glycol spacer (e.g., Spacer 9), hexa-ethylene glycol spacer (e.g., Spacer 18), a photocleavable moiety, or combinations or analogs thereof. Alternatively, or in addition, the cleavable moiety may be cleavable using one or more stimuli, e.g., photo-stimulus, chemical stimulus, thermal stimulus, etc.

The sequencing workflow 100 may not involve supports, for example when a template nucleic acid and/or its derivatives are directly attached to a substrate and amplified and/or sequenced from the substrate.

A template nucleic acid may include an insert sequence sourced from a biological sample. The template nucleic acid may be derived from any nucleic acid of the biological sample (e.g., endogenous nucleic acid) and result from any number of processing operations, such as but not limited to fragmentation, degradation or digestion, transposition, ligation, reverse transcription, extension, replication, etc. The template nucleic acid may be single-stranded, double-stranded, or partially double-stranded. A template nucleic acid may comprise one or more functional nucleic acid sequences. For example, the template nucleic acid may comprise a capture sequence, a primer sequence, a sequencing primer sequence, a barcode sequence, a sample index sequence, a unique molecular identifier (UMI), a flow cell adapter sequence, an adapter sequence, a target sequence, a random sequence, a binding sequence (e.g., for a splint, primer, template nucleic acid, capture sequence, etc.), or any other functional sequence useful for a downstream operation, a complement thereof, or any combination thereof. The template nucleic acid may comprise an adapter sequence configured to be captured by a capture sequence of an oligonucleotide coupled to a support. The one or more functional nucleic acid sequences may be disposed at one end or both ends of the insert sequence. A nucleic acid molecule comprising the insert sequence, or complement thereof, may be processed with (e.g., attached to, extend from, etc.) one or more adapter molecules to generate the template nucleic acid comprising the insert sequence and one or more functional nucleic acid sequences. A template nucleic acid may comprise one or more capture entities and/or one or more cleavable moieties that are described elsewhere herein.

Optionally, the supports and/or template nucleic acids may be pre-enriched (102). For example, a support comprising a distinct oligonucleotide sequence is pre-enriched to isolate from a mixture comprising support(s) that do not have the distinct oligonucleotide sequence. For example, a template nucleic acid comprising a distinct configuration (e.g., comprising a particular adapter sequence) is pre-enriched to isolate from a mixture comprising template nucleic acids that do not have the distinct configuration. In some cases, the capture entity on the supports and/or template nucleic acids are used for pre-enrichment.

The supports and template nucleic acids may be attached (103) to generate support-template complexes. A template nucleic acid may be coupled to a support via any method(s) that results in a stable association between the template nucleic acid and the support. For example, the template nucleic acid may hybridize to an oligonucleotide on the support; the template nucleic acid may be ligated to a nucleic acid coupled to the support; the template nucleic acid may hybridize to one or more intermediary molecules, such as a splint, bridge, and/or primer molecule, which hybridizes to an oligonucleotide on the support; and/or the template nucleic acid may be hybridized to an oligonucleotide on a support, which oligonucleotide comprises a primer sequence which is extended. In some cases, the respective concentrations of the supports and template nucleic acids may be adjusted such that a majority of support-template complexes are single template-attached supports (e.g., a support attached to a single template nucleic acid).

Optionally, support-template complexes may be pre-enriched (104), wherein a support-template complex is isolated from a mixture comprising support(s) and/or template nucleic acid(s) that are not attached to each other. In some cases, the capture entity on the supports and/or template nucleic acids are used for pre-enrichment.

The template nucleic acids may be subjected to amplification reactions (105) to generate a plurality of amplification products immobilized to the support. Such amplification reactions may comprise performing polymerase chain reaction (PCR) or any other amplification methods described herein, including but not limited to emulsion PCR (ePCR or emPCR), isothermal amplification, recombinase polymerase amplification (RPA), rolling circle amplification (RCA), multiple displacement amplification (MDA), bridge amplification, template walking, etc. Amplification reactions can occur while the support is immobilized to a substrate. Amplification reactions can occur off the substrate, such as in solution, or on a different surface or platform. Amplification reactions can occur in isolated reaction volumes, such as within multiple droplets in an emulsion during emulsion PCR (ePCR or emPCR), or in wells or tubes.

Optionally, subsequent to amplification, the supports, template nucleic acids, and/or support-template complexes may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. Nos. 10,900,078, U.S. Patent Pub. No. 20210079464A1, and International Patent Pub. No. WO2022040557A2, each of which is entirely incorporated by reference herein.

The template nucleic acids may be subject to sequencing (107). The template nucleic acid(s) may be sequenced while attached to the support. Alternatively, the template nucleic acid molecules may be free of the support when sequenced and/or analyzed. The template nucleic acids may be sequenced while immobilized to a substrate, such as via a support or otherwise. Examples of substrate-based sample processing systems are described elsewhere herein. Any sequencing method may be used, for example pyrosequencing, single molecule sequencing, sequencing by synthesis (SBS), sequencing by ligation, sequencing by binding, etc.

For example, sequencing comprises extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. Pub. No. 2022/0170089A1, which is entirely incorporated by reference herein for all purposes. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes).

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. The data analysis may comprise image processing, alignment to a genome or reference genome, training and/or trained algorithms, error correction, and the like.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed. The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates, detectors, and sample processing hardware that can be used in the sample processing system are described in further detail in U.S. Pat. Nos. 10,273,528, 10,267,790, 10,344,328, 10,512,911, 10,900,078, 11,268,143, 10,830,703, 10,852,518, and 11,155,868, U.S. Patent Pub. Nos. 20200326327A1, 20210079464A1, and 20210354126A1, and International Patent Pub. No. WO2022072652A1, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

In some examples, a substrate surface may comprise silicon. In some instances, a resist may be deposited on the substrate surface. The resist may be patterned by exposing the substrate surface to a dose of organosilane vapor. In some cases, the resist can be removed from the substrate surface before any patterning is performed. In some cases, the resist can be removed from the substrate surface after patterning. In some cases, no patterning is performed. In some cases, one or more cleaning steps may be performed before exposing the substrate surface to a dose of organosilane vapor to refine the resist definitions. The resist can be any resist known in the art. The resist may be a negative or positive resist. The resist may be a photo-curable resist. The resist may be a thermally curable resist. The resist may be applied to the substrate using methods known in the art. The applied resist may be used to create a mask on the substrate using various methods, such as photolithography methods, nanoimprint lithography (NIL), extended ultraviolet lithography (EUV), electron beam lithography, or optical lithography methods such as i-line stepper/scanner lithography, deep ultraviolet (DUV) lithography, extreme ultraviolet (EUV) lithography, and X-ray lithography.

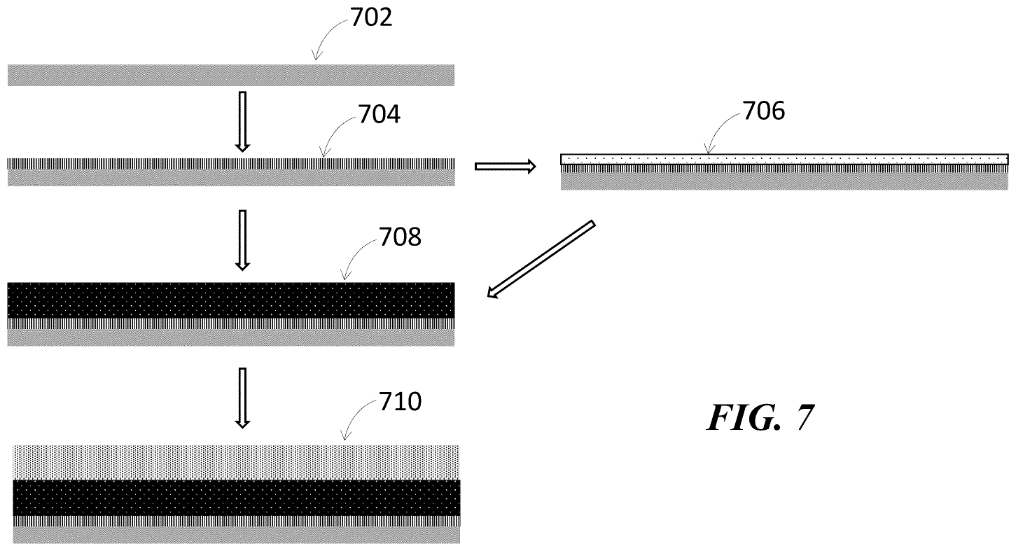

In an example, as shown in FIG. 7, the substrate surface may be processed by depositing and growing a silicon oxide layer 704 on a bare silicon wafer 702. Optionally, a hexamethyldisilazane (HMDS) layer 706 may be deposited. Additionally, or alternatively, a photoresist adhesion silane or bead passivation layer may be deposited. Optionally, a resist material 708 may be spin-coated onto the wafer. The resist may be a photoresist or a nanoimprint lithography photoresist. In some cases, the resist may be used to pattern the surface (e.g., via stepper photolithography or by imprinting and curing the resist with a NIL stamp). A resist layer may not be required to produce unpatterned substrate surfaces. Oxygen plasma may be used to descum any photoresist impurity (e.g., after any patterning), remove residual resist, and/or to activate the surface. A molecular coating of an organosilane 710 can be applied through a chemical vapor deposition (CVD) process. In some cases where patterning is performed, the resist may subsequently be removed from the wafer though sonication in a stripper solution known in the art to generate a substrate surface with a plurality of organosilane-functionalized locations.

The substrate may have the general form of a cylinder, a cylindrical shell or disk, a rectangular prism, or any other geometric form. The substrate may have a thickness (e.g., a minimum dimension) of at least and/or at most about 100 micrometers (μm), 200 μm, 500 μm, 1 millimeter (mm), 2 mm, 5 mm, 10 mm, 15 mm, 20 mm, 25 mm, 30 mm, 35 mm, 40 mm, 45 mm, 50 or mm. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least and/or at most about 1 mm, 2 mm, 5 mm, 10 mm, 20 mm, 30 mm, 40 mm, 50 mm, 100 mm, 150 mm, 200 mm, 300 mm, 400 mm, 500 mm, 1,000 mm, 1,500 mm, 2,000 mm, 2,500 mm, 3,000 mm, 4,000 mm, 5,000 mm or more.

One or more surfaces of the substrate may be exposed to and accessible from a surrounding open environment. In some cases, the surrounding open environment may be controlled and/or confined in a larger controlled environment.

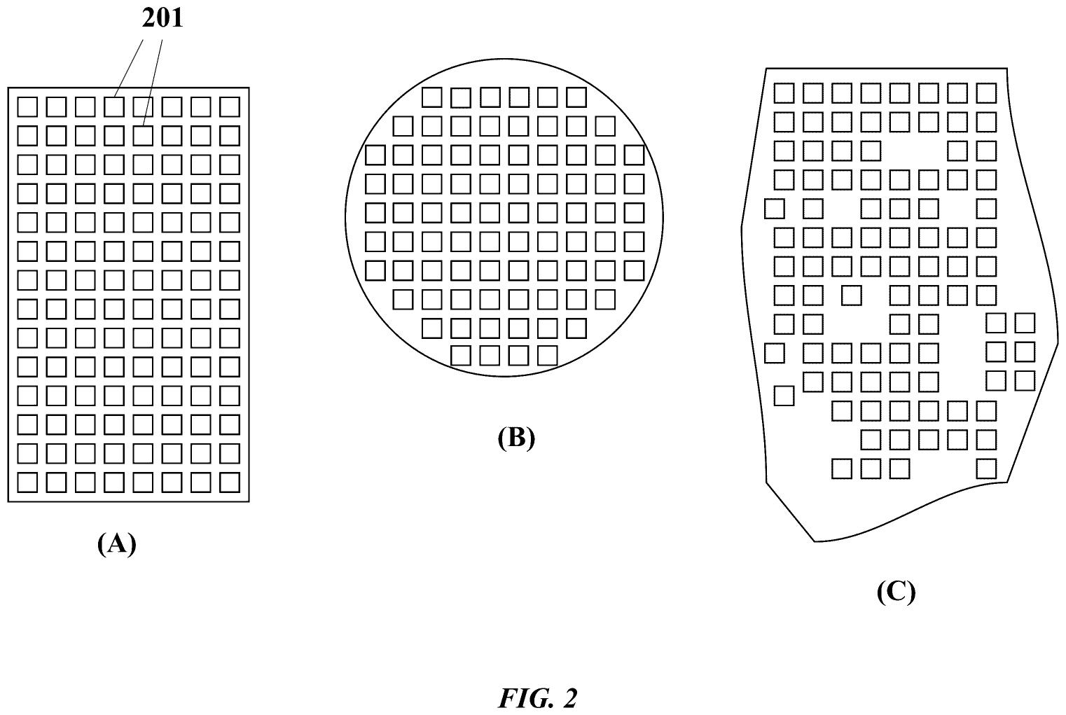

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing. In some cases, the individually addressable locations may be defined by physical features of the substrate (e.g., on a modified surface) to distinguish from each other and from non-individually addressable locations. In some cases, the individually addressable locations may not be defined by physical features of the substrate, and instead may be defined digitally (e.g., by indexing) and/or via the analytes and/or reagents that are loaded on the substrate (e.g., the locations in which analytes are immobilized on the substrate). The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays.

The substrate may have any number of individually addressable locations, for example, on the order of 1, 10¹, 10², 10³, 10⁴, 10⁵, 10⁶, 10⁷, 10⁸, 10⁹, 10¹⁰, 10¹¹, 10¹², 10¹³ or more individually addressable locations. Each individually addressable location may have any shape or form, for example the general shape or form of a circle, oval, square, rectangle, polygonal, or non-polygonal shape when viewed from the top. A plurality of individually addressable locations can have uniform shape or form, or different shapes or forms. An individually addressable location may have any size. In some cases, an individually addressable location may have an area of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 7, 8, 9, 10 square micron (μm²), or more. The individually addressable locations may be distributed on a substrate with a pitch determined by the distance between the center of a first location and the center of the closest or neighboring individually addressable location. Locations may be spaced with a pitch of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 micron (μm). In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of the locations, may be capable of immobilizing thereto an analyte (e.g., a nucleic acid, a protein, a carbohydrate, etc.) or a reagent (e.g., a nucleic acid, a probe molecule, a barcode molecule, an antibody molecule, a primer molecule, a bead, etc.). In some cases, an analyte or reagent may be immobilized to an individually addressable location via a support, such as a bead. In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location. A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.).

In some cases, an individually addressable location may comprise a distinct surface chemistry. The distinct surface chemistry may distinguish between different addressable locations and/or distinguish an individually addressable location from surrounding locations. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. A first location type or region type may comprise a positively charged surface chemistry and a second location type or region type may comprise a negatively charged surface chemistry. A first location type or region type may comprise a hydrophobic surface chemistry and a second location type or region type may comprise a hydrophilic surface chemistry. A first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. A combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, pillars, wells, cavities (e.g., micro-scale cavities or nano-scale cavities), and/or channels. The substrate may have regular textures and/or patterns across the surface of the substrate. The substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. The substrate may be textured or patterned such that all features are at or above a reference level of the surface (no features below a reference level of the surface, such as a well). The substrate may be textured or patterned such that all features are at or below a reference level of the surface (no features below a reference level of the surface, such as a pillar). In some instances, a texture of the substrate may comprise structures having a maximum dimension of at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. Alternatively, the substrate may be untextured and unpatterned.

A binder may be configured to immobilize an analyte or reagent to an individually addressable location. In some cases, a surface chemistry of an individually addressable location may comprise one or more binders. In some cases, a plurality of individually addressable locations may be coated with binders. The binders may be integral to the substrate. The binders may be added to the substrate. For instance, the binders may be added to the substrate as one or more coating layers. The substrate may comprise an order of magnitude of at least and/or at most about 10, 10², 10³, 10⁴, 10⁵, 10⁶, 10⁷, 10⁸, 10⁹, 10¹⁰, 10¹¹, 10¹², 10¹³ or more binders. The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. A single binder may bind a single analyte or single reagent, a single binder may bind a plurality of analytes or a plurality of reagents, or a plurality of binders may bind a single analyte or a single reagent. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis, referred to herein as a rotational axis. The rotational axis may or may not be an axis through the center of the substrate. The systems, devices, and apparatus described herein may further comprise an automated or manual rotational unit configured to rotate the substrate. The rotational unit may comprise a motor and/or a rotor. For instance, the substrate may be affixed to a chuck (such as a vacuum chuck). The substrate may be rotated at a rotational speed of at least about 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with different rotational velocities during different operations described herein, for example with higher velocities during reagent dispense and with lower velocities during analyte loading and imaging operations. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For example, bead loading may be performed with controlled dispensing. For controlled dispensing, the substrate may be rotated with a rotational frequency of no more than 60, 50, 40, 30, 25, 20, 15, 14, 13, 12, 11, 10, 9, 8, 7, 6, 5, 4, 3, 2, 1 rpm or less. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, low speed for analyte or reagent incubation, etc.).

In some cases, the substrate may be movable in any vector or direction. For example, such motion may be non-linear (e.g., in rotation about an axis), linear (e.g., on a rail track), or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. Alternatively, some nozzles may share a fluidic line or fluidic valve, such as for pre-dispense mixing and/or to dispensing to multiple locations. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. The nozzles may be angled towards or away from a center of the substrate, or not angled (e.g., normal to the substrate plane). Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed onto or adjacent to the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least and/or at most about 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. One or more conditions may be applied to attain a film thickness that is within a range defined by any two of the preceding values. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate. The substrate may then be subjected to vibration, which may spread the reagent to different locations across the substrate. Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

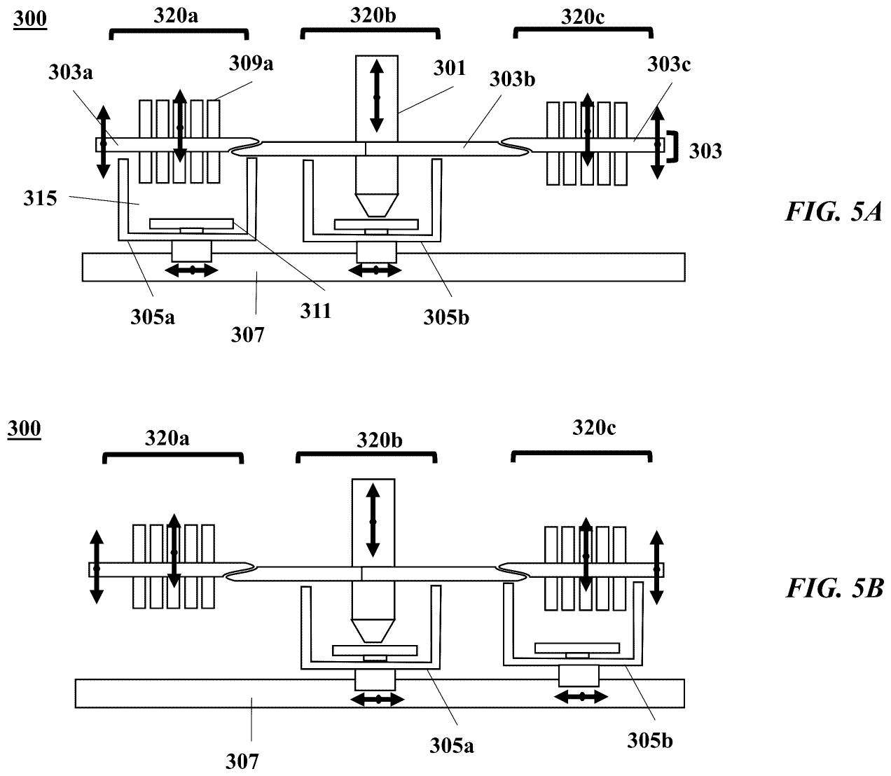

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. Two or more solutions or reagents may be delivered to the substrate using the same or different delivery methods. Two or more solutions may be delivered to the substrate such that the time between contacting a solution or reagent and a subsequent solution or reagent is substantially similar for each region of the substrate contacted to the one or more solutions or reagents. A solution or reagent may be delivered as a single mixture. The solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. Dispensing of each component of the two or more components may be temporally separated. Dispensing of each component may be performed using the same or different delivery methods. Direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, and the composition of the fluid may be adjusted such that the layer of fluid is maintained during incubation. In some instances, during incubation, the substrate may be rotated at a rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotated with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

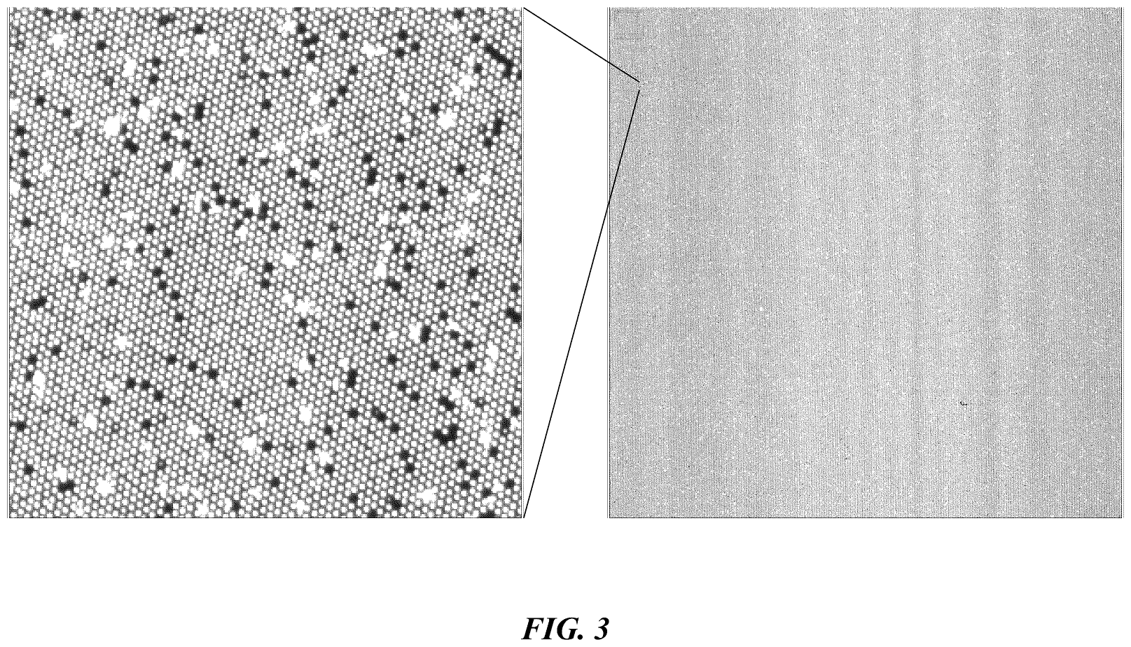

A sample may comprise beads, as described elsewhere herein, for example beads comprising nucleic acid colonies bound thereto. In some cases, an order of magnitude of at least and/or at most about 10¹, 10², 10³, 10⁴, 10⁵, 10⁶, 10⁷, 10⁸, 10⁹, 10¹⁰, 10¹¹, 10¹², 10¹³ or more beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag (e.g., barcode) that identifies a bead amongst a plurality of beads. FIG. 3 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. After sample loading, a “bead occupancy” may generally refer to the number of a type of individually addressable locations comprising at least one bead out of the total number of individually addressable locations of the same type. A bead “landing efficiency” may generally refer to the number of beads that bind to the surface out of the total number of beads dispensed on the surface.

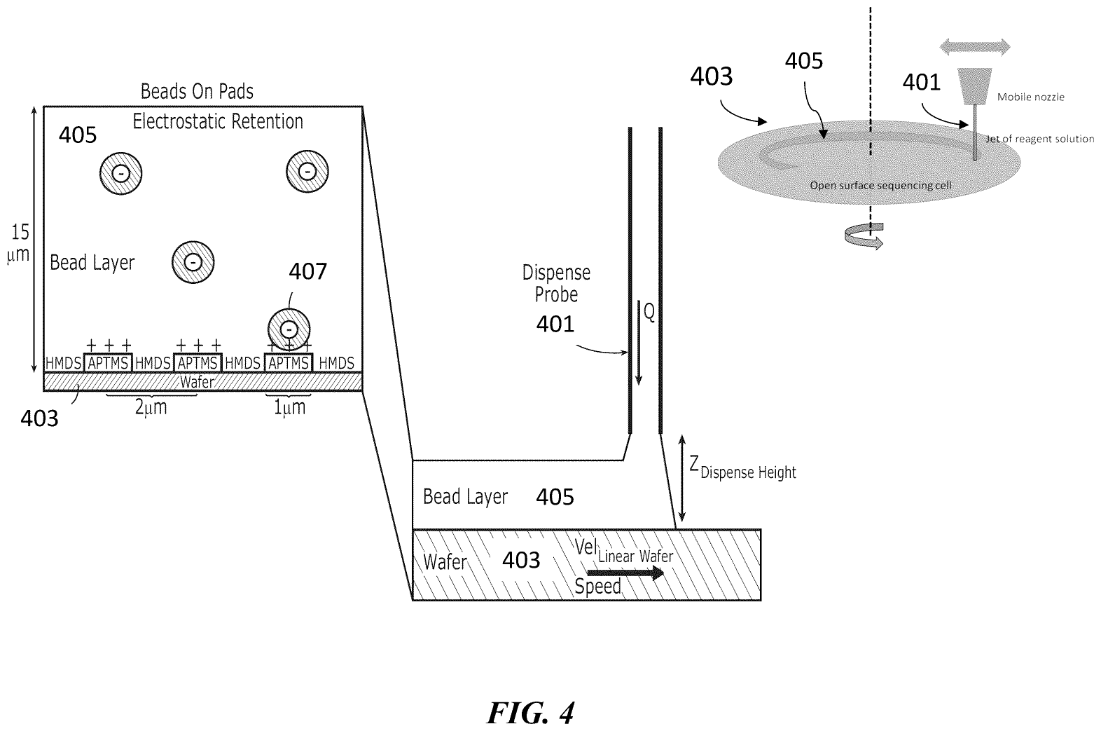

In some cases, beads may be dispensed to the substrate according to one or more systems and methods shown in FIG. 4. As shown in FIG. 4, a solution comprising beads may be dispensed from a dispense probe 401 (e.g., a nozzle) to a substrate 403 (e.g., a wafer) to form a layer 405. The dispense probe may be positioned at a height (“Z”) above the substrate. In the illustrated example, the beads are retained in the layer 405 by electrostatic retention and may immobilize to the substrate at respective individually addressable locations. A set of beads in the solution may each comprise a population of amplified products (e.g., nucleic acid molecules) immobilized thereto, which amplified products accumulate to a negative charge on the bead. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 405 a bead may successfully land on a first location of the first location type (as in 407). In the illustrated example, the location size is 1 micron, the pitch between the different locations of the same location type (e.g., first location type) is 2 microns, and the layer has a depth of 15 micron. The top right panel illustrates that a reagent solution may be dispensed from the dispense probe 401 as the layer 405 along a path on an open surface of the substrate 403. The reagent may be dispensed on the surface in any desired pattern or path. The substrate 403 and the dispense probe 401 may move in any configuration with respect to each other to achieve any pattern (e.g., linear pattern, substantially spiral pattern, etc.).

Dispense mechanisms described herein may be operated by a fluid flow unit which may be controlled by one or more controllers, individually or collectively. The fluid flow unit may comprise any of the hardware and software components described with respect to the dispense mechanisms herein.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A signal may be an optical signal (e.g., fluorescent signal), electronic signal, or any detectable signal. The signal may be detected during rotation of the substrate or following termination of the rotation. The signal may be detected while the analyte is in fluid contact with a solution. The signal may be detected following washing of the solution. In some instances, after the detection, the signal may be muted, such as by cleaving a label from a probe and/or the analyte, and/or modifying the probe and/or the analyte. Such cleaving and/or modification may be performed by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in multiple cycles and for each iteration, the additional signals detected may provide incremental, or final, data about the analyte during the processing. For example, where the analyte is a nucleic acid molecule and the processing is sequencing, additional signals detected for each iteration may be indicative of one or more bases in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, tube lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any one or more optical sources (e.g., lasers, LED light sources, etc.). In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise one or more controllers operatively coupled to the one or more sensors, individually or collectively programmed to process optical signals from the one or more sensors, such as for each region of the rotating substrate.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the immersion fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate. In some cases, the immersion fluid may be continuously replenished or recycled via an inlet and outlet to the enclosure.

### High Throughput

An open substrate may be processed within a modular local sample processing environment. A barrier comprising a fluid barrier may be maintained between a sample processing environment and an exterior environment during certain processing operations, such as reagent dispensing and detecting. Barrier systems are described in further detail in U.S. Pat. No. 10,512,911, which is entirely incorporated herein by reference. Systems and methods comprising a fluid barrier are described in further detail in U.S. Patent Pub. No. 20210354126A1, which is entirely incorporated herein by reference. A modular local sample processing environment may be defined by a chamber and a lid plate, where the lid plate is not in contact with the chamber, and the gap between the lid plate and the chamber may comprise the fluid barrier. The fluid barrier may comprise fluid (e.g., air) from the sample processing environment and/or the exterior environment and may have lower pressure than the sample processing environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least and/or at most 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

An open substrate may be retained in the same or approximately the same physical location during processing of an analyte and subsequent detection of a signal associated with the processed analyte. Alternatively, different operations on or with the open substrate may be performed in different stations disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. The open substrate may transition between different stations by transporting the sample processing environment comprising the chamber containing the open substrate between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity. In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

One or more modular sample environment systems (each having its own barrier system, e.g., fluid barrier) can be used between the different stations. In some instances, the systems described herein may be scaled up to include two or more of a same station type. For example, a sequencing system may include multiple processing and/or detection stations. FIGS. 5A-5B illustrate a system 300 that multiplexes two modular sample environment systems in a three-station system. In FIG. 5A, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 5B, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 (e.g., lid plate) defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 5A, and this re-stationing can be repeated (e.g., between the configurations of FIGS. 5A and 5B) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations substantially simultaneously while the detection station is kept idle.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Self-Assembly of Beads on Substrates

Provided herein are devices, systems, methods, compositions, and kits that allow for self-assembly of supports (e.g., beads) on substrates. Such devices, systems, methods, compositions, and kits can be applied alternatively or in addition to the operations described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein. In some instances, the devices, systems, methods, compositions, and kits described herein are useful in loading a plurality of beads adjacent to a substrate, such that the plurality of beads may self-assemble into a monolayer adjacent to (e.g., on) the substrate. Each bead of the plurality of beads or a subset of the plurality of beads may be individually addressable.

In an aspect, disclosed herein is a method for self-assembly of a plurality of beads adjacent to (e.g., on or under) a substrate. Such a method may comprise providing a substrate and a plurality of beads, dispensing the beads adjacent to the substrate, and subjecting the substrate or the plurality of beads to conditions sufficient for self-assembly of the plurality of beads adjacent to the substrate. In some instances, the beads may self-assemble on a substrate that is unpatterned or substantially unpatterned. Such a method may obviate the need for one or more substrate pre-processing operations (e.g., patterning, etching), thereby allowing for facile generation of bead-laden substrates. The bead-laden substrates may comprise substantially uniform monolayers of densely packed arrays of beads that allow for downstream processing (e.g., sequencing), as is described elsewhere herein.

Substrates: The substrate may be a solid or semi-solid substrate. As is described elsewhere herein, the substrate may comprise or partially comprise any useful material, such as silicon, glass, metal, polymer, etc. The substrate may be planar or substantially planar, e.g., having a planarity at a micrometer scale, at a nanometer scale, or at smaller scales, as described herein. The substrate may be unpatterned or substantially unpatterned. For example, an unpatterned substrate may lack regular or defined topographical features (e.g., wells, posts, grooves, troughs, hills, pillars, etc.). An unpatterned substrate may lack any defined or discernable patterns; for example, the unpatterned substrate may lack defined regions that are spatially, chemically, or mechanically distinct. The unpatterned substrate may be substantially uniform. Alternatively or in addition to, the unpatterned substrate may comprise one or more artefacts or defects (e.g., from the manufacturing process) that may occur in commercially available unpatterned substrates. For example, the substrate may comprise a silicon wafer and may comprise crystalline defects or random irregularities, e.g., slight vacancies, flaking, texturing that occur as a product of the manufacturing. In some instances, the substrate is a silicon wafer. Alternatively or in addition, the substrate may comprise a silicon oxide layer. In some instances, the substrate is a glass wafer, optionally comprising a liquid crystal display.

Beads: The plurality of beads (e.g., supports as described elsewhere herein) may be made of any useful composition. The beads may be solid or semi-solid. The beads may be formed from a metal, a ceramic, a polymer, glass, rubber, or other material or a combination thereof. The beads may be a semi-solid material, such as a gel, e.g., a hydrogel. The gel may comprise one or more polymeric materials, which may be natural or synthetic. The gel may comprise, in non-limiting examples, hyaluronic acid, chitosan, heparin, alginate, fibrin, polyacrylamide, polyvinyl alcohol, polyethylene glycol, or combinations or derivatives thereof. The beads may be electrostatically charged (e.g., positively charged or negatively charged), which, in some examples, may allow for electrostatic interactions between the bead and the substrate. In some instances, the beads may be negatively charged and the substrate, or a portion thereof (e.g., a surface or portions of a surface of the substrate) may be positively charged. Alternatively, the beads may be positively charged, and the substrate or a portion thereof may be negatively charged. The beads may be magnetic or comprise magnetic components. The beads may comprise one or more linkers or chemical moieties, which may allow for covalent attachment of the bead to the substrate. For example, the bead may comprise a first reactive moiety, such as an azide moiety, an alkyne moiety, a phosphorothioate moiety, an iodide moiety, an amine moiety, or a phosphate moiety, a nitrone moiety, an alkene moiety, a thioate moiety, etc., and the substrate may comprise a second reactive moiety, such that reaction of the first reactive moiety and the second reactive moiety may result in formation of a bond. Such a reaction may comprise subjecting the reactive moieties to suitable conditions such as a suitable temperature, pH, pressure, light, or providing a reagent or catalyst, e.g., copper, ruthenium, etc. The first reactive moiety and the second reactive moiety may be linked via “click” chemistry, which beneficially may allow for a stronger interaction (e.g., formation of covalent bonds) between the beads and the substrate in a controlled manner (see Example 5).

As described herein, the plurality of beads may be coupled to one or more nucleic acid molecules. The plurality of beads may be coupled to a deoxyribonucleic acid (DNA) molecule or a ribonucleic acid (RNA) molecule, or a hybrid DNA-RNA molecule. The nucleic acid molecules may be double-stranded, partially double-stranded, or single-stranded. The nucleic acid molecules may be nucleic acid nanoballs (e.g., resulting from rolling circle amplification). A bead may comprise, coupled thereto, one type of nucleic acid molecule (e.g., a population of identical nucleic acid molecule) or multiple types of nucleic acid molecules (e.g., comprising at least two non-identical nucleic acid molecules). In some instances, the plurality of beads may comprise template nucleic acid molecules coupled thereto. The template nucleic acid molecules may be derived from a sample and may comprise, for example, an analyte (e.g., mRNA, cDNA or derivatives thereof). Such template nucleic acid molecules may be the same or different across beads. For example, a first bead may comprise template nucleic acid molecules derived from a first sample, and a second bead may comprise template nucleic acid molecules derived from a second sample different than the first sample. Alternatively or in addition to, beads of the plurality of beads may comprise template nucleic acid molecules from the same sample. The nucleic acid molecules coupled to the beads may comprise other useful sequences, e.g., primer sequences, sequencing primers, barcode sequences, unique molecular identifiers, restriction sites, transposition sites, noncanonical nucleotides, etc., as is described elsewhere herein. In some instances, the nucleic acid molecules coupled to the beads may comprise an optical tag or moiety, e.g., a fluorescence label, a dye, etc., as is described elsewhere herein.

The plurality of beads may be any useful size or range of sizes. For example, beads of the plurality of beads may be about 0.01 micrometers, 0.05 micrometers, 0.1 micrometers, 0.5 micrometers, 1 micrometer, 5 micrometers, 10 micrometers, 50 micrometers, or greater in diameter. The beads may be at most about 50 micrometers, at most about 10 micrometers, at most about 5 micrometers, at most about 1 micrometer, at most about 0.5 micrometers, at most about 0.1 micrometers, at most about 0.05 micrometers, at most about 0.01 micrometers or smaller in diameter. The beads may comprise a range of sizes, for example from about 0.1 micrometers to about 10 micrometers in diameter, or from about 1 micrometer to about 5 micrometers in diameter, etc. In some cases, the bead diameter may comprise an average diameter of beads in the plurality of beads. In some cases, at least 50%, 55%, 60%, 65%, 70%, 75%, 80%, 85%, 90%, 95%, 99% or more of beads in the plurality of beads comprise the average diameter.

The plurality of beads may be provided in a solution or as part of a suspension. The solution or suspension may comprise any useful components, including but not limited to buffering agents, salts, enzymes, proteins, peptides, amino acids, chelators, lipids, carbohydrates, metals, nucleic acid molecules, nucleotides, ions, stabilizing agents, preservatives, etc. In some instances, the solution or suspension may comprise one or more proteins that may interact with the nucleic acid molecules of the bead, e.g., single-stranded binding proteins. For example, a bead may comprise a single-stranded template nucleic acid molecule (e.g., DNA or RNA) and may be provided in a solution comprising a single-stranded binding protein. Examples of methods, systems, and compositions for bead-solution mixtures, e.g., comprising single-stranded binding proteins can be found in International Pub. No. WO2022/051296, which is incorporated by reference herein in its entirety.

Self-assembly: The plurality of beads may be dispensed adjacent to a substrate, and the plurality of beads or the substrate may be subjected to conditions sufficient for self-assembly of the beads adjacent to the substrate. Such dispensing and self-assembly may comprise any number of operations. In an example, the plurality of beads may be provided in a solution and dispensed, in a drop-wise manner, to the substrate. Subsequent to the dispensing, the plurality of beads may be incubated or allowed to spread adjacent to the substrate, e.g., across a surface of the substrate. Self-assembly may occur during the dispersion or spreading process and may be facilitated by modulating one or more operating parameters, such as the dispensed volume of the solution, the concentration of the beads in the solution, the incubation time, or additional chemistry operations or additives in the solution. In some instances, the self-assembly may occur without application of external force; for example, the beads may assemble naturally through evaporative or capillary forces, or other non-covalent interactions. Self-assembly of the beads may result in a substantially uniform layer (e.g., a monolayer) of the beads adjacent to the substrate. Such a substantially uniform monolayer may be characterized, for example, by a bead-to-bead tolerance of approximately twice the diameter of the plurality of beads. For example, the bead-to-bead tolerance may fall in a range of a distance (e.g., 1 bead-diameter in any direction) with respect to the average plane of beads. The beads may be distributed on a substrate with a pitch determined by the distance between the center of a first bead and the center of the closest or neighboring bead. In some instances, the self-assembled, substantially uniform monolayer may be arranged such that individual beads of the plurality of beads are individually addressable, e.g., distinguishable by the naked eye, by microscopy, or by image recognition or image processing methods.

In some instances, the dispersion of the beads across the substrate may be assisted by providing an applied force, e.g., a shear force, a centrifugal force, or other applied force. In one example, following dispensing of the plurality of beads adjacent to (e.g., on a surface of) the substrate, the substrate may be rotated to disperse the plurality of beads across the surface of the substrate. The substrate may be rotated at any useful rotational velocity, e.g., about 1 revolution per minute (rpm), about 10 rpms, about 50 rpms, about 100 rpms, about 500 rpms, about 1000 rpms, about 5,000 rpms, about 10,000 rpms or greater. The substrate may be rotated in a range of rotational velocities, e.g., from about 500 rpms to about 8,000 rpms. The substrate may be rotated for any useful or duration of time, e.g., for about 1 s, about 5s, about 10 s, about 20 s, about 30 s, about 40 s, about 50 s, about 1 min, about 5 min, about 10 min, or longer. The substrate may be rotated within a range of duration, e.g., from about 15 to about 30 seconds. In some instances, the dispersion of the beads across the substrate via rotation may facilitate even distribution of the beads across the substrate, which may aid in generating substantially uniform, self-assembled monolayers of the plurality of beads.

In some instances, the dispensing of the plurality of beads adjacent to (e.g., on or across) the substrate may occur by translating the substrate relative to a solution or suspension comprising the plurality of beads, or alternatively, by translating the solution or suspension comprising the plurality of beads relative to the substrate. For example, the plurality of beads may be provided in a solution in a container, and the substrate may be contacted with the solution, e.g., by translating the substrate vertically into and out of the solution (e.g., using a dipping method). The translation of the substrate may occur in any direction, e.g., substantially vertically, substantially horizontally, or at an angle. The substrate may be contacted with the plurality of the beads (e.g., in the solution in the container) for any useful duration of time, e.g., for about 1 s, about 10 s, about 20 s, about 30 s, about 40 s, about 50 s, about 1 minute, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, or more. The contacting may occur for a range of durations, e.g., between about 1 minute and 60 minutes, between about 60 minutes and 120 minutes, etc. Subsequent to the contacting, the substrate may be translated relative to the solution or suspension (e.g., removed from a container comprising the solution or suspension) and optionally incubated, as described above. The incubation may occur at any useful temperature and duration and may facilitate drying of the plurality of beads adjacent to the substrate.

At any useful or convenient time, the substrate and the plurality of beads may be incubated, e.g., to allow for dispersion or self-assembly. For example, the substrate and the plurality of beads may be incubated subsequent to the dispensing of the plurality of beads, or following application of a force (e.g., centrifugal force) to disperse or spread the beads across the substrate. The incubation may occur at any useful temperature and for any useful period of time. The incubation may occur at a temperature above or below ambient temperature. For example, incubation of the substrate and the plurality of beads may occur at ambient temperature or at an elevated temperature in order to facilitate drying of the plurality of the beads on the substrate. The incubation may occur at a temperature of about 0 degrees Celsius, at about 10 degrees Celsius, at about 20 degrees Celsius, at about 30 degrees Celsius, at about 40 degrees Celsius, at about 50 degrees Celsius, at about 60 degrees Celsius, at about 70 degrees Celsius, at about 80 degrees Celsius, at about 90 degrees Celsius, at about 100 degrees Celsius, at about 150 degrees Celsius, at about 200 degrees Celsius or higher. The incubation may occur within a range of temperatures, e.g., from about 25 degrees Celsius to about 100 degrees Celsius. The incubation may occur for about 1 second (s), about 10 s, about 20 s, about 30 s, about 40 s, about 50 s, about 1 minute, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, or more. The incubation time may occur in a range of durations, e.g., from about 5 minutes to about 40 minutes.

The beads may be provided at any useful concentration and volume. For example, the beads may be provided in a solution at a concentration of about 1 bead/microliter (μL), about 10 beads/μL, about 100 beads/μL, about 1000 beads/μL, about 10,000 beads/μL, about 100,000 beads/μL, about 1,000,000 beads/μL, about 10,000,000 beads/μL, about 100,000,000 beads/μL or greater. A range of concentrations of the beads may be provided, e.g., between about 10,000,000 beads/μL and 20,000,000 beads/μL. The beads may be provided at any useful volume, depending on the processing. For example, the beads may be provided in a solution of about 1 μL, 10 μL, 100 μL, 1 milliliter (mL), 10 mL, 100 mL, 1000 mL, or greater. The volume of solution provided may vary depending on the processing method. For example, in instances where the substrate is translated in a container comprising the solution comprising the beads, a greater volume (e.g., about 200 mL or greater) may be provided. In another example, in instances where the beads are provided drop-wise to the substrate, volumes of about 20 microliters to 40 microliters may be provided.

The substrate may be subjected to one or more conditions sufficient to dry or desiccate the substrate or the plurality of beads. For example, the plurality of beads may be provided in a solution or suspension, and the solution or suspension may be dispensed adjacent to (e.g., on or across a surface of) the substrate. Optionally, the substrate may be rotated to facilitate the dispersion of the plurality of beads adjacent to the substrate. Prior to, during, or subsequent to the dispensing of the plurality of beads adjacent to the substrate, the substrate, the plurality of beads, or both the substrate and the plurality of beads may be subjected to conditions sufficient to desiccate or dry the substrate or the plurality of beads. Such drying or desiccation may comprise incubating the substrate or the plurality of beads at any suitable temperature. For example, the substrate or the plurality of beads may be heated to facilitate evaporation of the solution. Alternatively or in addition, drying or desiccation may be performed by other approaches, e.g., providing an air stream (e.g., nitrogen or oxygen gas) and directing the air stream toward or at an angle to the substrate or plurality of beads, exposure to vacuum, use of a desiccation chamber, etc. The drying or desiccation may be performed for any suitable duration of time, e.g., for about 1 s, about 10 s, about 20 s, about 30 s, about 40 s, about 50 s, about 1 minute, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, or more. The drying or desiccation may occur for a range of durations, e.g., between about 1 minute and 60 minutes, between about 15 minutes and 30 minutes, etc.

Sequencing accuracy depends upon the ability of a detector or post-detection analysis to differentiate between adjacent beads (e.g., to be able to resolve individual beads and hance distinct nucleic acid template sequences). There is an inherent tradeoff between increasing sequencing throughput (e.g., by increasing density) and retaining high detection accuracy. Bead self-assembly can However, the presence of bead aggregations (and hence decreased detection ability) can negate any benefits from increased density if there is not a corresponding increase in detection resolution capabilities. Therefore, in some cases, it may be beneficial to be able to increase the average pitch between beads in a self-assembled scheme. To this end, non-sequencing beads (or other spacing particles) that will not inhibit self-assembly can be used in combination with sequencing beads (e.g., beads comprising nucleic acid molecules).

By way of example, in some instances, the plurality of beads to be loaded onto a substrate may comprise a first subset of beads coupled to nucleic acid molecules and a second subset of beads not coupled to nucleic acid molecules. The subset of beads that lack nucleic acid molecules coupled thereto may help prevent overcrowding of beads that are coupled to nucleic acid molecules after loading onto a substrate (e.g., may increase the pitch between beads comprising nucleic acid molecules after self-assembly). In some cases, the first subset of beads comprises at least 50%, 60%, 70%, 75%, 80%, 85%, 90%, 99%, 99.5%, or 99.9% of the plurality of beads. That is, in such cases, the second subset of beads comprises at most 50%, 40%, 30%, 25%, 20%, 15%, 10%, 5%, 1.5%, or 0.1% of the plurality of beads. In some cases, as the percentage of the second subset of beads increases, the pitch between beads in the first subset of beads increases.

Substrate pre-processing: In some instances, the substrate may be processed prior to dispensing of the plurality of beads adjacent to the substrate. For example, the substrate may be wetted using a buffer. In such instances, the substrate may be contacted with a pre-wetting buffer. The pre-wetting buffer may be useful, for example, in changing a surface property of the substrate, e.g., the hydrophilicity or hydrophobicity, charge, ionic concentration, or other property, or for priming the coupling of the plurality of beads to the substrate. The pre-wetting buffer may comprise an ionic buffer. The ionic buffer may comprise magnesium, e.g., magnesium chloride, at any useful molar concentration, e.g., about 1 micromolar, 100 micromolar, 1 millimolar (mM), 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1M, or greater. The magnesium salt molar concentration may fall in a range of concentrations, e.g., between about 10 mM and about 50 mM. Additional examples of pre-wetting buffers and methods and systems comprising pre-wetting butters can be found in International Pub. No. WO 2022/051296, which is incorporated by reference herein in its entirety.

In some instances, the substrate may be functionalized or adsorbed with one or more surface moieties, e.g., to promote binding or coupling of the plurality of beads to the substrate. For example, the one or more surface moieties may comprise a silane group, such as an organosilane (see FIG. 7). The silane group may be an amino silane such as 3-aminopropyltriethoyxsilane (APTES), 3-aminopropyltrimethoyxsilane (APTMS), 3-(Ethoxydimethylsilyl)propylamine, Bis[3-(trimethoxysilyl)propyl]amine, 3-mercaptopropyltrimethoxysilane (MPTS), octadecyltrichlorosilane (OTS), octadecyltrimethoxysilane (OTMS), polytetrafluoroethylene organosilanes (PFS) etc. The silane group may be deposited or applied to the substrate using any useful approach, e.g., solution deposition, chemical vapor deposition, gas phase evaporation, etc.

In some instances, the substrate may be cleaned, e.g., prior to functionalization or adsorption of a surface moiety (e.g., silane). Such cleaning may comprise, in non-limiting examples, washing with acetone, isopropanol, water, piranha, UV irradiation, UV treatment, ozone treatment, oxygen plasma treatment, etc.

Any number of pre-processing operations may be performed on the substrate, e.g., prior to dispensing of the plurality of beads adjacent to the substrate, and in any order. For example, in some examples, it may be advantageous to clean the substrate, followed by vapor deposition of a silane (e.g., APTES or APTMS) adjacent to the substrate, followed by wetting using an ionic buffer. In some instances, the substrate may be treated with a silane and then wetted. Alternatively, the substrate may be treated with silane, optionally rinsed, and then contacted with the plurality of beads directly.

Shrinking: The plurality of beads may be subjected to conditions sufficient to shrink or decrease the size of the plurality of beads, e.g., subsequent to or concurrent with self-assembly of the plurality of beads adjacent to (e.g., on or across) the substrate. Alternatively or in addition, the surface area of the substrate that is not in contact with a bead of the plurality of beads may be increased. In some examples, the uncontacted surface area of the substrate may be increased without increasing the average center-to-center distance between beads of the plurality of beads. A change or difference in the ratio of the bead-contacting areas and the uncontacted surface area may be achieved using, for example, a shrinking buffer, which may decrease the size of the plurality of beads. In some instances, the shrinking buffer may be provided in a solution comprising the plurality of beads and co-dispensed with the plurality of beads adjacent to the substrate. Alternatively or in addition, the shrinking buffer may be provided subsequent to the dispensing or self-assembly of the plurality of beads adjacent to the substrate. The shrinking buffer may comprise reagents sufficient to decrease the size of beads of the plurality of beads. In some examples, the shrinking buffer comprises a polymer, such as polyethylene glycol (PEG). The PEG may be provided at any useful molar mass, e.g., PEG 100 (g/mol), PEG 200, PEG 300, PEG 400, PEG 500, PEG 600, PEG 700, PEG 800, PEG 900, PEG 1000, PEG 2000, PEG 3000, PEG 4000, PEG 5000, PEG 6000, PEG 7000, PEG 8000, PEG 9000, PEG 10000, or higher. The PEG may be provided at a range of molar masses, e.g., from about 4000 g/mol to about 8000 g/mol. The PEG may be provided at any useful concentration. For example, the PEG may be provided at a 0.1% w/v, 1% w/v, 2% w/v, 3% w/v, 4% w/v, 5% w/v, 6% w/v, 7% w/v, 8% w/v, 9% w/v, 10% w/v, 20% w/v, 30% w/v, 40% w/v, 50% w/v, 60% w/v, 70% w/v, 80% w/v, 90% w/v, or greater. The PEG may be provided at a range of concentrations, e.g., between about 9% w/v and 12% w/v, or about 1% w/v to about 1.5% w/v.

The shrinking buffer may comprise one or more salts, such as magnesium salts, including, but not limited to, magnesium chloride, magnesium sulfate, magnesium glycinate, magnesium citrate, magnesium oxide, etc. For example, the shrinking buffer may comprise magnesium salt at any useful molar concentration, e.g., about 1 micromolar, 100 micromolar, 1 millimolar (mM), 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1M, or greater. The magnesium salt molar concentration may fall within a range of concentrations, e.g., between about 5 mM and about 50 mM.

The shrinking buffer may comprise one or more organic cations. In some instances, the shrinking buffer comprises a polyamine. Examples of polyamines include, in non-limiting examples, alkyl polyamines, diethylenetriamine, triethylenetetramine, macrocyclic polyamines, 1,4,7-triazacyclononane, cyclen, cyclam, Tris (2-aminoethyl)amine, spermidine, spermine, thermospermine. In some instances, the shrinking buffer comprises spermine. In some cases, spermine may have nitrogen atoms replacing carbon atoms one or more of positions 1, 5, 10, and 14 of a polyazaalkane. In some cases, spermine may have a nitrogen atom replacing a carbon atom at one or more of positions 1, 2, 3, 4, 5, 6, 7, 8, 9, 10, 11, 12, 13, or 14 of a polyazaalkane. In some cases, spermine may be charged or carry a charge. In some cases, a charged spermine or spermine carrying a charge may be spermine ion. In some cases, spermine ions may comprise spermine¹⁺, spermine²⁺, spermine³⁺, spermine⁴⁺, or a combination thereof. In some case, the shrinking buffer solution comprising spermine may also comprise a salt derivative of spermine.

The organic cations (e.g., spermine) may be provided at any useful molar concentration, e.g., about 1 micromolar, 100 micromolar, 1 millimolar (mM), 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1M, or greater. The organic cations may fall in a range of concentrations, e.g., between about 5 mM and about 50 mM.

Alternatively or in addition, a change or difference in the ratio of the bead-contacting areas and the uncontacted surface area may be achieved using other approaches, for example, the application of heat, which can, in some instances, decrease the size of the beads, increase the non-contacted surface area of the substrate, or both.

Sequencing: The methods, kits, systems, and compositions provided herein may be useful in processing nucleic acid molecules, e.g., via sequencing. In some instances, as described herein, one or more nucleic acid molecules (e.g., template nucleic acid molecules) may be provided on a bead of the plurality of beads, and the plurality of beads may be dispensed on a substrate and allowed to self-assemble. Subsequent to self-assembly of the plurality of beads, the one or more nucleic acid molecules may be sequenced. The sequencing may comprise the use of one or more nucleotide flow sequences, as is described elsewhere herein. Such flow sequencing may comprise (i) providing a nucleotide-containing reagent to the plurality of beads or said substrate and (ii) detecting the nucleotide. Operations (i) and (ii) may be repeated, optionally with a different nucleotide and optionally with one or more intervening washing flows.

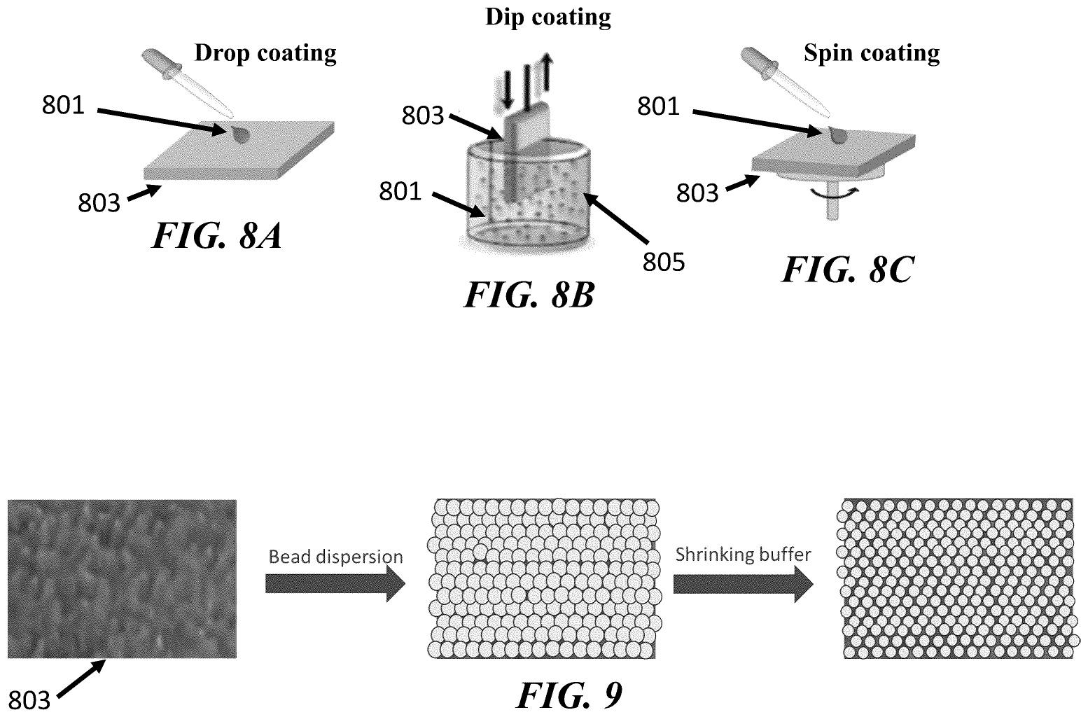

Workflows: FIGS. 8A-8C shows an example schematic of a variety of workflows for obtaining self-assembled beads adjacent to a substrate, and further processing of the plurality of beads or substrate. FIG. 8A schematically shows a drop-coating method, in which a plurality of beads is provided in a solution 801, which may be dispensed drop-wise on a substrate 803. The solution 801 may be provided in sufficient quantity to cover the substrate 803 in the solution 801. The solution 801 may be provided in a quantity that is sufficient to cover an area less than the entire substrate 803. FIG. 8B schematically shows a dip-coating method, in which a plurality of beads is provided in a solution 801 in container 805. Substrate 803 may be translated relative to container 805 (shown in FIG. 8B as a vertical translation). That is, the substrate 803 may be translated while the container 805 is stationary or alternatively, the container 805 may be translated while the substrate 803 is stationary. Translation may be performed horizontally or vertically. The entire surface area of substrate 803 may be contacted with solution 801 (e.g., the substrate may be submerged entirely in the solution in the container). Alternatively, a portion less than whole of the substrate may be contacted with solution 801 (e.g., only the portion of the substrate may be submerged in the solution in the container). FIG. 8C schematically shows a spin coating method, in which a plurality of beads is provided in a solution 801, which may be dispensed drop-wise on a substrate 803. The substrate 803 may be rotated prior to, during, or subsequent to the dispensing of the solution 801 adjacent to (e.g., on or across) the substrate 803. Optionally, the substrate 803 may be rotated such that at least a portion of the substrate is contacted with the solution (e.g., a portion greater than an initial portion of the substrate contacted with the solution via drop-wise addition). Optionally, at least 50%, at least 60%, at least 70%, at least 80%, at least 90%, at least 95%, at least 99%, or at least 100% of the surface area of substrate may be contacted with the solution during or subsequent to the rotation of the substrate. FIG. 9 schematically illustrates the surface area of substrate 803 subsequent to dispersion of a plurality of beads adjacent to the substrate (i.e., via solution 801).

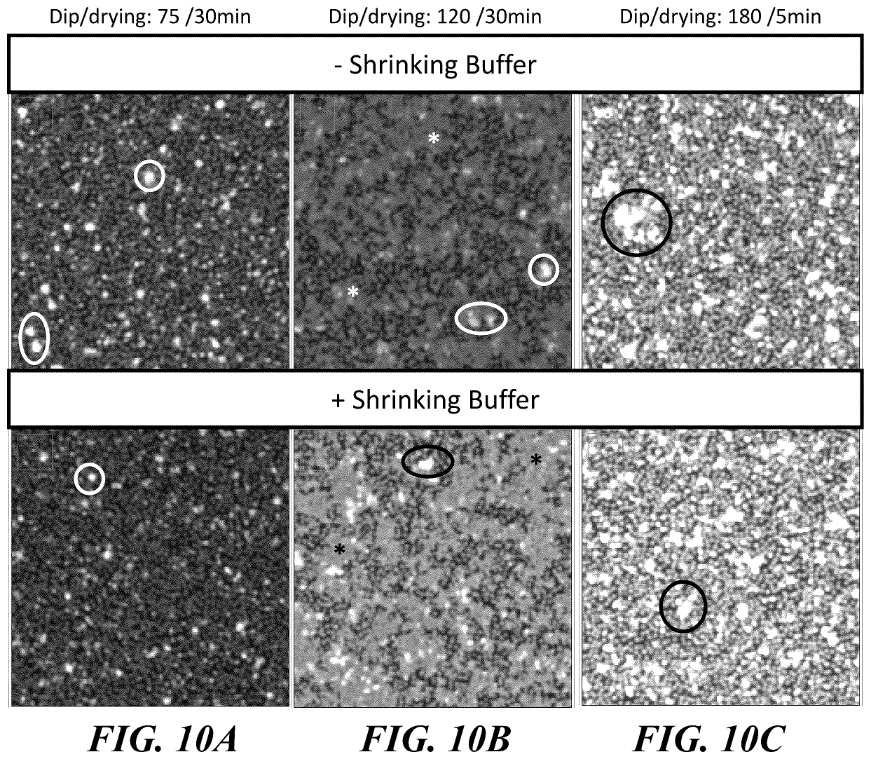

An unpatterned substrate 803 may be provided. Alternatively, the substrate may be partially patterned. In some cases, a partially patterned substrate comprises a plurality of features, where the features are smaller than the beads provided for self-assembly on or adjacent to the substrate (e.g., a nanopatterned substrate). For example, the beads may have an average diameter of ˜1 μm and the features may have an average diameter of 10-50 nm. In such cases, a bead in the plurality of beads may contact two or more features after loading and/or self-assembly. Optionally, the unpatterned or partially patterned substrate 803 may be contacted with one or more reagents, e.g., an amino silane (e.g., APTES or APTMS), which may facilitate adhesion of the plurality of beads to a surface of the substrate 803. In some instances, the substrate may be subjected to wetting, e.g., via contacting with a wetting buffer (e.g., comprising magnesium chloride) prior to contacting the substrate with the plurality of beads. The plurality of beads may comprise a plurality of nucleic acid molecules coupled thereto and may be dispensed on the substrate, e.g., using any of the methods provided in FIGS. 8A-8C, optionally dried (e.g., via incubating the substrate at an elevated temperature), and contacted with a shrinking buffer. The shrinking buffer may decrease the size of the plurality of the beads, which may aid in improving access to the individual beads or nucleic acid molecules coupled thereto. Advantageously, such a decrease in the size of the plurality of beads may also improve imaging (e.g., microscopy) of the substrate or the individual beads and thus improve downstream sequencing analysis. See also, FIGS. 10-13, and Example 1.

Also provided herein, in some aspects, are kits for facilitating self-assembly of beads on a substrate. Such a kit may comprise a substrate, which may be unpatterned and substantially planar, a plurality of beads, and instructions for forming a self-assembled monolayer of the plurality of beads adjacent to the substrate. The substrate, as described herein, may be a solid or semi-solid substrate. Similarly, the plurality of beads may be solid or semi-solid beads and may, in some instances, comprise a plurality of nucleic acid molecules (e.g., DNA molecules) coupled thereto. The DNA molecules may be single-stranded or double-stranded. The kit may comprise additional elements which may be useful in generating the self-assembled monolayer, e.g., a pre-wetting buffer, such as those disclosed herein (e.g., an ionic buffer, a buffer comprising magnesium). In some instances, the kit may comprise a shrinking buffer, which may comprise PEG, magnesium salts (e.g., magnesium chloride), or other reagents, as is described elsewhere herein.

Also disclosed herein, in some aspects, are systems for self-assembly of beads on a substrate. Such a system may comprise a substrate, which may be unpatterned and substantially planar, and a plurality of beads. The system may be configured such that at least a first subset of the plurality of beads is in a substantially close-packed configuration and at least a second subset of the plurality of beads is in a substantially monolayer configuration. The first subset and the second subset may be different or may be the same or substantially the same. The close-packed configuration may comprise, for example a center-to-center distance between neighboring beads of a defined distance, e.g., about 100 nanometers (nm), about 500 nm, about 1 micrometer (μm), about 2 μm, about 3 μm or greater. In some instances, the close-packed configuration comprises a center-to-center distance between neighboring distance at a range of distances, e.g., from about 1 μm to about 1.8 μm. See, Example 1.

The substrate, as described herein, may be a solid or semi-solid substrate. Similarly, beads in the plurality of beads may be solid or semi-solid beads and may, in some instances, comprise a plurality of nucleic acid molecules (e.g., DNA molecules) coupled thereto. The DNA molecules may be single-stranded or double-stranded. The kit may comprise additional elements which may be useful in generating the self-assembled monolayer, e.g., a pre-wetting buffer, such as those disclosed herein (e.g., an ionic buffer, a buffer comprising magnesium). In some instances, the kit may comprise a shrinking buffer, which may comprise PEG, magnesium salts (e.g., magnesium chloride), or other reagents, as is described elsewhere herein.

Methods: Provided herein are different methods that comprise self-assembly of beads on a substrate. The methods may comprise one or more of (i) loading beads onto the substrate for self-assembly, (ii) amplification of analytes (e.g., nucleic acid molecules) on beads while the beads are on or off the substrate in which self-assembly of the beads occurs, and (iii) sequencing beads on or off the substrate in which self-assembly of the beads occurs.

A method may comprise loading a plurality of beads onto a substrate for self-assembly, wherein the plurality of beads are post-amplification beads. The post-amplification beads may comprise a plurality of amplified products immobilized thereto. The method may comprise, prior to loading, subjecting analytes to amplification to generate the post-amplification beads comprising the plurality of amplified products immobilized thereto. Subsequent to self-assembly of the beads on the substrate, the amplified products may be sequenced while immobilized to the substrate. In some cases, the method may comprise, subsequent to self-assembly of the beads on the substrate, subjecting the amplified products to one or more additional amplification procedures while the beads are immobilized to the substrate to perform on-surface amplification, such as to generate a plurality of second stage amplified products (e.g., second stage concatemers or second stage extension products). The second stage amplified products may be immobilized to the beads. The second stage amplified products may be immobilized off the beads such as directly to the substrate or to other objects (e.g., additional beads) coupled to the substrate. For example, the substrate may comprise surface primers or other objects that comprise surface primers which can be extended during the one or more additional amplification procedures. The surface primers or other objects on the substrate may be arranged in a patterned or unpatterned manner. The second stage and/or additional stage amplified products may be generated and/or sequenced. In an example, the post-amplification beads comprise ePCR amplification products, in which a bead comprises a colony of identical or substantially identical nucleic acid molecules. In an example, the post-amplification beads comprise RCA amplification products, in which a bead comprises a concatemer molecule comprising multiple copies of a template nucleic acid sequence (or segments thereof).

A method may comprise loading a plurality of beads loaded onto a substrate for self-assembly, wherein the plurality of beads are pre-amplification beads. The pre-amplification beads generally refer to beads that have not been subjected to amplification procedures. In some cases, the pre-amplification beads that are loaded may comprise a plurality of surface primers. In some cases, the plurality of surface primers may comprise a single type of primer (e.g., forward primers or reverse primers). In some cases, the plurality of surface primers may comprise two types of primers (e.g., forward primers and reverse primers). In some cases, the plurality of surface primers may comprise more than two types of primers (e.g., comprising more than two types of primer sequences). In some cases, the pre-amplification beads that are loaded may comprise template nucleic acid molecules. In some cases, a pre-amplification bead comprises a single template nucleic acid molecule. In some cases, a pre-amplification bead comprises at most single template nucleic acid molecule. For example, bead-single template nucleic acid molecule complexes may be pre-enriched prior to loading, as described elsewhere herein. In some cases, a pre-amplification bead comprises multiple template nucleic acid molecules. A template nucleic acid molecule may be attached to a pre-amplification bead covalently or non-covalently. For example, a template nucleic acid molecule may be hybridized to a surface primer of a bead. In another example, a complement of the template nucleic acid molecule may be hybridized to a surface primer of the bead and the surface primer extended to generate the template nucleic acid molecule. Alternatively, the pre-amplification beads that are loaded may not comprise template nucleic acid molecules, and the template nucleic acid molecules may be dispensed to the beads on the substrate during or after self-assembly to attach the template nucleic acid molecules to the beads. The pre-amplification beads may self-assemble on the substrate and subjected to one or more amplification procedures while the beads are immobilized to the substrate to perform on-surface amplification, to generate a plurality of amplified products (e.g., concatemers or extension products). Any amplification method may be used, such as RCA, RPA, MDA, bridge amplification, etc. The amplified products may be immobilized to the beads. The amplified products may be immobilized off the beads such as directly to the substrate or to other objects (e.g., additional beads) coupled to the substrate. For example, the substrate may comprise surface primers or other objects that comprise surface primers which can be extended during the one or more amplification procedures. The surface primers or other objects on the substrate may be arranged in a patterned or unpatterned manner. In some cases, second stage and/or additional stage amplified products may be generated by performing additional amplification procedures on the substrate. Subsequent to amplification, the amplified products may be sequenced while immobilized to the substrate. Alternatively, the amplified products may be removed from the substrate, loaded onto an additional substrate, and sequenced on the additional substrate.

### Computer Systems

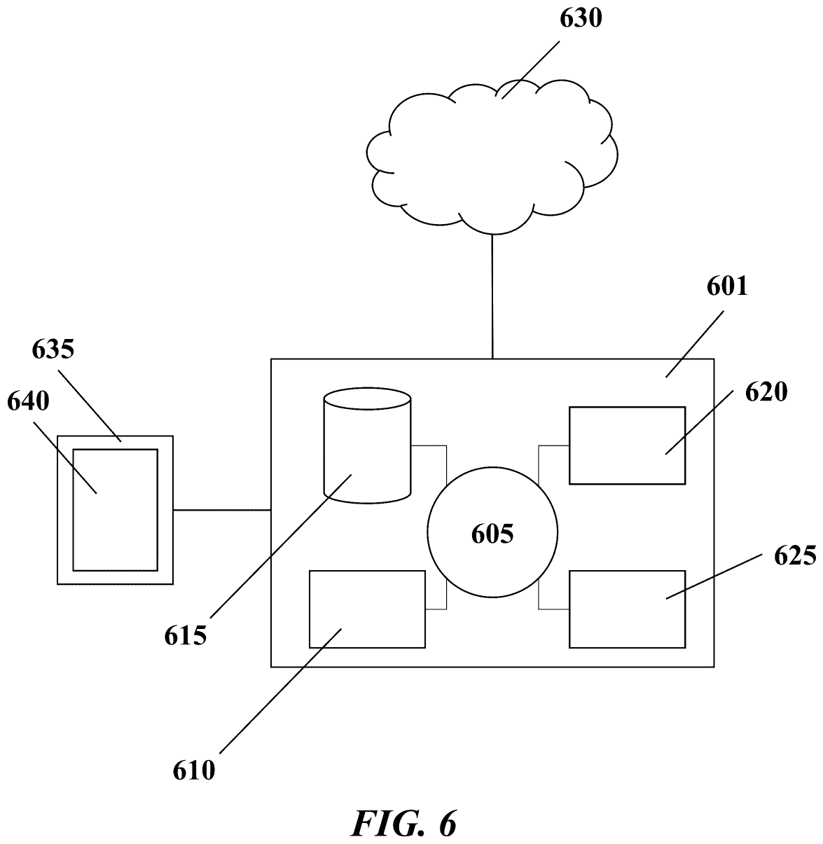

The present disclosure provides computer control systems that are programmed to implement methods of the disclosure. FIG. 6 shows a computer system 601 that is programmed or otherwise configured to implement methods of the disclosure, such as to control the systems described herein (e.g., reagent dispensing, detecting, etc.) and collect, receive, and/or analyze sequencing and/or image information. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an isolated or substantially isolated internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server. The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback. The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple®iPad, Samsung® Galaxy Tab), telephones, Smart phones (e.g., Apple®iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610. The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 835 that comprises a user interface (UI) 640 for providing, for example, images (e.g., micrographs) of the substrates or the plurality of beads, along with the analysis of the images (e.g., pitch, spacing, occupancy, intensity, nucleic acid sequence data, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, determine the occupancy, spacing, or other parameters (e.g., full-width half-maximum, mean fluorescence intensity) of an image (e.g., micrograph of a bead or plurality of beads on or adjacent to a substrate).

### EXAMPLES

### Example 1: Generating Monolayers of Beads on an Unpatterned Substrate

As described herein, a substrate (e.g., an unpatterned substrate) may be contacted with a plurality of beads to generate a self-assembled monolayer of the beads adjacent to the substrate. A substrate, such as a silicon wafer may be provided. The silicon wafer may optionally comprise a silicon dioxide layer. The silicon wafer may be treated with a silane, e.g., 3-aminopropyltrimethoxysilane (APTMS). Representative images of beads dispersed along the substrate, for each condition described below, were obtained using a 10× microscope objective.

A) Dip-coating: A dip-coating process may be performed, in which the wafer is contacted in a container with a solution comprising a plurality of beads (e.g., hydrogel beads) comprising nucleic acid molecules coupled thereto (e.g., as illustrated in FIG. 8B). A substrate may be deposited into the container (e.g., a test tube, a beaker, etc.), thereby contacting the beads with at least a portion of the substrate. Alternatively or additionally, the substrate can be translated with respect to the container. The substrate may be incubated in the container for any useful duration of time, removed from the container, and then subjected to drying (e.g., by placing in a temperature-controlled chamber).

FIGS. 10A-10CB show example results of beads applied to a substrate via dip-coating of the substrate. These experiments were performed under conditions in which a plurality of beads (e.g., hydrogel beads), with an average diameter of 0.5 micrometers and comprising single stranded (e.g., unamplified) nucleic acid molecules coupled thereto, were provided to the substrate in a solution at a concentration of 15 million beads per microliter. The bead solution was provided in a container (e.g., tube) at a volume of 10 milliliters. The substrate was placed horizontally in the container. The substrate was then incubated in the container with the beads for a first time: 75 min, 120 min, or 180 min (e.g., ‘dip’ times as indicated, respectively, in FIGS. 10A, 10B, and 10C). Subsequently, the substrate was removed from the container and subjected to drying for a second time (e.g., 30 min, 30 min, and 5 min, respectively for FIGS. 10A, 10B, and 10C).

In the top panel of each of FIGS. 10A-10C the substrates were not exposed to a shrinking buffer (e.g., subsequently to beads being dispersed onto the substrates). In contrast, in the lower panel of each of FIGS. 10A-10C the substrates were exposed to a shrinking buffer (e.g., subsequent to beads being dispensed onto the substrates). In each panel, examples of bead aggregation are indicated by circles and examples of unfocused regions are indicated by asterisks (*). Regions that are not focused are indicative of beads that lie above or below the focal plane (e.g., beads are not within a monolayer that is uniform or substantially uniform along the substrate). Qualitative analysis of each panel suggests that beads were successfully dispensed and dispersed along the substrates. Indeed, FIG. 10A in particular exemplifies substrates with fairly uniform distribution of beads; however, each tested condition also demonstrated aggregation and/or non-monolayer behavior of the dispensed beads.

B) Drop-coating: FIGS. 11A-11D show example results of beads applied to substrates via drop-coating in which the wafer is contacted with an aliquot of a solution comprising a plurality of beads (e.g., as illustrated in FIG. 8A). These experiments were performed under conditions in which a plurality of beads (e.g., ISP-5HG beads—ThermoFisher's Ion sphere particles (ISPs)), comprising single-stranded nucleic acid molecules coupled thereto (and labeled with FAM to facilitate fluorescent imaging) were provided in solution at a concentration of 20 million beads per microliter. Prior to beads being dispensed onto substrates via drop-coating, each substrate was exposed to a pre-wetting buffer to facilitate bead loading. See Example 2. The pre-wetting buffer comprised 10 mM Tris pH7, 0.05% Tergitol, and 50 mM MgCl₂ (“TTM50”).

The bead solution was then dispensed on the substrate in a drop-wise fashion in a volume of 20 microliters and allowed to spread across the substrate, thus permitting beads to disperse along the substrate. The substrate was then incubated at room temperature for varying durations of time (e.g., 45 min, 60 min, 75 min, or 90 min, respectively for FIGS. 11A-11D), thus providing time for beads to become associated with the substrate and for the buffer of the bead solution to evaporate.

In the lower panels of each of FIGS. 11A-11D, a shrinking buffer was then applied to the substrate. The shrinking buffer (“WB24”) comprised 10% w/v PEG8000 and 50 mM MgCl₂ (e.g., in 20 mM Tris pH 8.8, 0.08M NaCl, and 0.1% Triton X-100). In each panel, examples of bead aggregation are indicated by circles and examples of unfocused regions are indicated by asterisks (*). Regions that are not focused are indicative of beads that lie above or below the focal plane (e.g., beads are not within a monolayer that is uniform or substantially uniform along the substrate). Qualitative analysis of each panel suggests that beads were successfully dispensed and dispersed along the substrates. However, FIG. 11C in particular illustrates regions that are out of focus. In FIG. 11D the beads appear much smaller than in the other figures, indicating that the beads themselves were dried (e.g., decreased substantially in diameter). Both FIGS. 11A and 11B demonstrate well-dispersed beads along the substrate (e.g., individual beads are distinguishable), with only minor aggregations.

The substrates in FIGS. 11A and 11B were further evaluated. The average occupancy of the substrates was determined based on a 1.8 micron pitch, which was the average center-to-center bead distance. The full-width half maximum (“FWHM” in microns) of the beads were determined based on measured fluorescence intensity. The shrinking percentage was based upon comparison between the condition when no shrinking buffer was applied (upper panels) and the condition where shrinking buffer was applied (lower panels). The results are detailed in Table 1.

TABLE 1
Results of Drop-coating ISP beads, with pre-wetting using TT50M

| | | Full-width | % bead shrinkage |
|---|---|---|---|
| | | Half-maximum | (e.g., compared |
| Drying time \| | | (FWHM) | with no |
| +/−shrinking | Average % | (e.g., bead | shrinking |
| buffer | occupancy | diameter (μm)) | buffer condition) |
| 45 minutes drying \| | 84% | 1.1 | — |
| −shrinking buffer | | | |
| 45 minutes drying \| | 84% | 0.96 | 13% |
| +shrinking buffer | | | |
| 60 minutes drying \| | 91% | 1.14 | — |
| −shrinking buffer | | | |
| 60 minutes drying \| | 91% | 0.99 | 13% |
| +shrinking buffer | | | |

As shown in Table 1, for the substrate with a 45-min drying time (e.g., FIG. 11A), the average occupancy was about 84%, with a bead FWHM of 1.1 microns without shrinking buffer and 0.96 microns with the shrinking buffer. For the substrate with a 60-min drying time (e.g., FIG. 11B), the average occupancy was about 91%, with a bead FWHM of 1.14 microns without shrinking buffer and 0.99 microns with the shrinking buffer. In each case, there was approximately a 13% decrease in the bead size due to the application of the shrinking buffer (e.g., as shown in the lower panels of FIGS. 11A-11B). A full hour of drying resulted in a higher average percent occupancy of the substrate space (i.e., an improved utilization of substrate surface area), and in both cases addition of the shrinking buffer after drying resulted in a substantial decrease in FWHM (i.e., decreasing the difficulty in image analysis for potential subsequent sequencing reactions).

C) Spin-coating: FIGS. 12A and 12B show example results of applying beads via spin-coating to substrates in which the wafer is contacted with an aliquot of a solution comprising a plurality of beads during rotation of the substrate (e.g., as illustrated in FIG. 8C). These experiments were performed under conditions in which a plurality of beads (e.g., ISPs), comprising double-stranded nucleic acid molecules coupled thereto were provided in solution at a concentration of 25 million beads per microliter. Prior to beads being dispensed onto substrates, each substrate (e.g., an unpatterned silicon wafer) was exposed to a pre-wetting buffer to facilitate bead loading. See Example 2. The pre-wetting buffer comprised 10 mM Tris pH7, 0.05% Tergitol, and 50 mM MgCl₂ (e.g., “TTM50”). The beads were then dispensed in a volume of 16 μL. The substrate was incubated for 75 minutes. After incubation, the substrate was then rotated at 5000 rpm for a second time (e.g., 5 sec) while 200 μL of buffer solution was dispensed. The rotation and exposure to buffer solution was repeated three times. The substrate was then cured with SYBR gold (e.g., to permit subsequent fluorescent analysis of the beads) and incubated at room temperature for 10 minutes. For addition of shrinking buffer, the substrate was washed with 200 μL of shrinking buffer and then incubated for 3 minutes. The shrinking buffer comprised 10% PEG8000, 50 mM MgCl₂ (e.g., in 20 nM Tris pH 8.8, 0.08M NaCl, and 0.1% Triton X-100).

In each figure, percent occupancy was determined using a 1.4 μm pitch size (e.g., the average center-to-center distance between beads). For FIG. 12A, in which no shrinking buffer was applied, the average percent occupancy was 92%. For FIG. 12B, in which shrinking buffer was applied, the average percent occupancy was 93%. In each case, the beads were observed in a relatively uniform monolayer (with some minor bead aggregations). FIG. 12B, in which the shrinking buffer was applied, there was a qualitative decrease in bead size (i.e., increase in resolution of individual beads).

Overall, these data indicate that the methods provided herein (e.g., especially drop-coating and spin-coating) are useful for generating a substrate comprising, adjacent thereto, a plurality of beads that are self-assembled in a relatively uniform monolayer. The application of a shrinking buffer further serves to decrease the size of the beads. Such shrinking may beneficially allow for improved downstream data processing by facilitating detection of individual beads (e.g., analysis of images obtained during sequencing and subsequent sequence determination).

It will be appreciated that the listed volumes, concentrations, durations, number of repetitions, buffer components, rotational velocities, etc. are provided by way of example only and that different quantities or ranges of these values may be used. Moreover, the operations may be performed in any useful or convenient order. For example, drying or incubation may be performed following dispensing of the beads, following rotation cycles, following application of the shrinking buffer, etc. Additional operations may also be performed, for example, staining (e.g., using a nucleic acid stain such as SYBR gold or other fluorescent markers), imaging, sequencing, etc.

### Example 2: Incubating the Substrate with Mg²⁺ Prior to Bead Loading Promotes High Occupancy of Beads without Aggregation

Incubation of a substrate with cations, such as divalent cations including Mg²⁺ or Ca²⁺, encourages high occupancy of beads on the substrate and minimizes bead aggregation. Although cations promote bead aggregation when the beads themselves are exposed to the ions for prolonged periods (e.g., such as when beads are incubated in solution with a cation-containing buffer prior to loading on substrates), the same cations also advantageously facilitate dense packing and immobilization of the beads onto small features (i.e., micrometer level features) of the substrate. Aggregation and dense packing are inherently related effects of loading beads onto a substrate. One goal with bead loading is to minimize aggregation, which degrades the sequencing information obtainable from a loaded substrate, while still permitting dense packing, which increases sequencing efficiency. The cations themselves can screen the high, negative charges of the beads and reduce bead-bead repulsion that occurs on substrates with small feature sizes.

To investigate the effectiveness of cations in promoting surface occupancy while still minimizing bead aggregation, wafers with a pitch size of 1.8 μm were incubated (prewet) with a TT (10 mM of Tris, pH=7.0; 0.055% Tergitol) buffer containing various amounts of Mg²⁺ (50 mM, 100 mM, 200 mM, and 300 mM) for 0.5-1 minute. The beads were incubated with the TT buffer that lacked Mg²⁺ for 60 minutes in a sample tube prior to being dispensed onto the wafer. As a control, both the wafer and beads were incubated with a TTM buffer (10 mM of Tris, pH=7.0; 0.055% Tergitol; 10 mM MgCl₂) for 0.5-1 minutes and 60 minutes, respectively. Table 2 summarizes the resulting average substrate occupancy percentage on the wafers with the beads in different experiments.

TABLE 2
Coupon wafer occupancy with beads on a coupon
wafer with a pitch size of 1.8 μm

| | Bead | Average |
|---|---|---|
| | incubation | substrate |
| Coupon wafer incubation buffer | buffer | occupancy (%) |
| TTM buffer | TTM buffer | 94 |
| TT buffer + 50 mM Mg²⁺ (TT50M) | TT buffer | 91 |
| TT buffer + 100 mM Mg²⁺ (TT100M) | TT buffer | 92 |
| TT buffer + 200 mM Mg²⁺ (TT200M) | TT buffer | 92 |
| TT buffer + 300 mM Mg²⁺ (TT300M) | TT buffer | 94 |

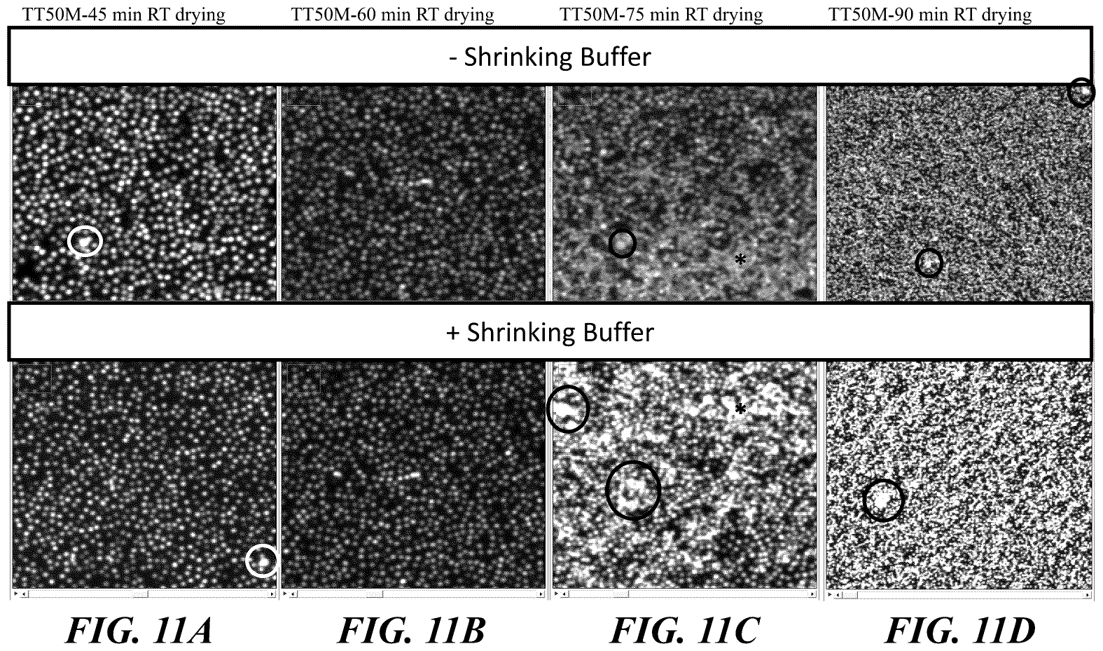

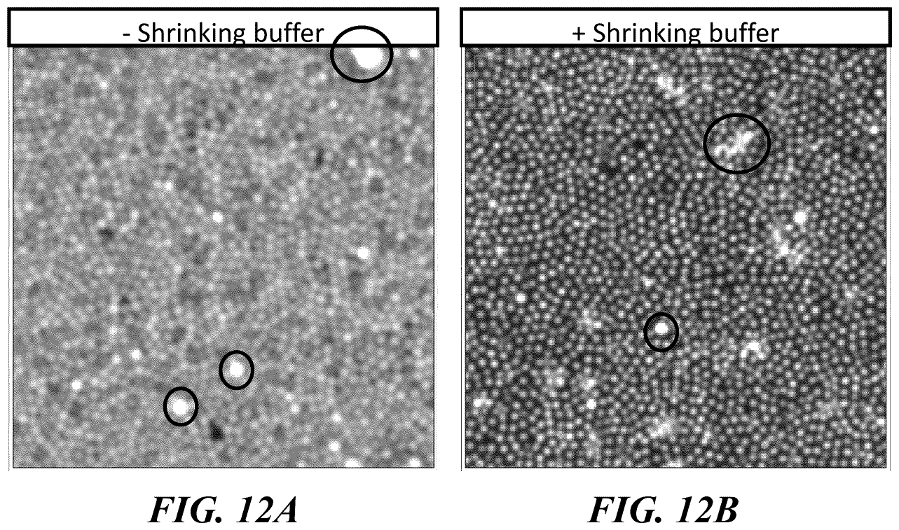

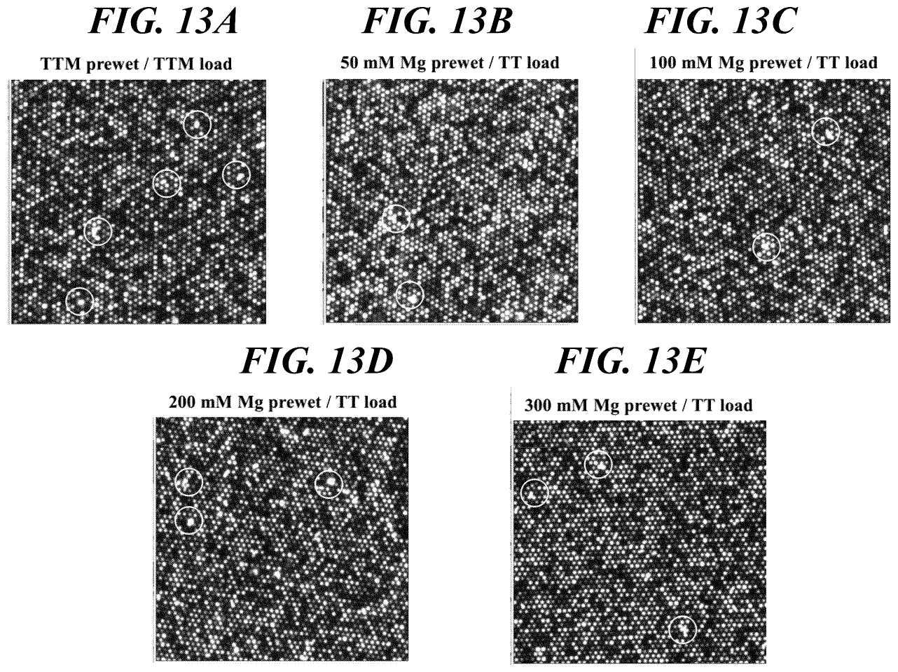

Substrate occupancy measures how much area or how many locations (i.e., micrometer level features) of the substrate are covered with beads. It does not inform on bead aggregation, but rather success of bead loading. As shown in FIGS. 13A-13E, high average substrate occupancies were achieved in the wafers incubated with various concentrations of Mg²⁺. FIGS. 13B-13E show the loading of beads on wafers incubated with 50 mM Mg², 100 mM Mg², 200 mM Mg², and 300 mM Mg², respectively. The beads incubated in buffer without any Mg²⁺ (i.e., when the beads were incubated with the TT buffer) showed significantly less bead aggregation on wafer as compared to the control experiment (FIG. 13A) where the beads and the wafers were both incubated in buffer containing Mg²⁺ (i.e., TTM buffer).

Even when the beads were exposed to the same total amount of Mg²⁺ (e.g., through either incubation of the beads in buffer containing Mg²⁺ or from prewetting a wafer in buffer containing Mg²⁺), incubation in Mg²⁺ containing buffer resulted in aggregates while Mg²⁺ prewetting discouraged bead aggregation without affecting the substrate occupancy. Thus, the timing (i.e., on or off wafer) of bead exposure to Mg²⁺ is surprisingly important. Table 3 summarizes average substrate occupancy when beads were incubated with or without buffer containing Mg²⁺ (e.g., the TT buffer or the TTM buffer, respectively), while the wafer was incubated with TT buffer containing Mg²⁺ (at either 50 mM or 100 mM) for 0 min. The wafers in this experiment had an average pitch size of 1.5 μm.

TABLE 3
Coupon wafer occupancy with beads on a coupon wafer
with a pitch size of 1.5 μm

| | | Average |
|---|---|---|
| Coupon wafer | Bead incubation | substrate |
| incubation buffer | buffer | occupancy (%) |
| TT buffer + 50 mM Mg²⁺ | TT buffer + 50 mM Mg²⁺ | 88 |
| (TT50M) | (TT50M) | |
| TT buffer + 100 mM Mg²⁺ | TT buffer | 88 |
| (TT100M) | | |

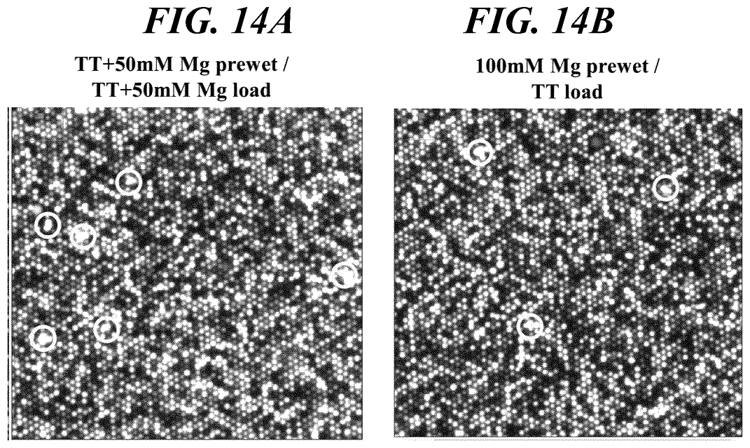

A high occupancy of beads on the substrates was achieved in either scenario. In addition, as shown in FIGS. 14A and 14B, the beads incubated without any Mg²⁺ (i.e., when the beads were incubated with the TT buffer) showed significantly less bead aggregation compared to that of the control, even though the beads were exposed to the same amounts of Mg²⁺ in total in both experiments.

Therefore, it can be concluded that exposure of beads themselves to cations encourages bead aggregation, but that incubation (prewetting) of the substrate with the same cations promotes substrate occupancy without promoting the bead aggregation. Hence, one way to reduce the bead aggregation effect is to minimize the exposure of beads to cations before dispensing onto a substrate. The benefits of dense loading of beads onto wafers can be achieved by incubating the wafers (i.e., alone without the beads, prior to loading) with cations.

### Example 3: Using a Shrinking Buffer after Bead Loading to Decrease Bead Size

Various polymers or cations were tested for providing better resolution of individual particles during imaging (e.g., detection of individual particles after particles were loaded onto substrates). Particles such as ISP-5HG beads (ThermoFisher's Ion sphere particles (ISPs)) were prepared (e.g., attached or annealed to) DNA molecules amplified by emulsion PCR (emPCR). The beads were dispensed onto substrates (e.g., silicon wafers) for imaging, where the dispensing was performed using typical loading buffers (e.g., 20 mM Tris pH 8.8, 0.08M NaCl and 0.1% Triton X-100). Prior to imaging, wafers were exposed to buffers comprising one or more of the various polymers or cations to be tested. The size of beads (e.g., measured as full-width, half-max (FWHM)) was determined by using a detector during or subsequent to imaging. Specifically, the bead sizes were determined based on images obtained from fluorescent imaging.

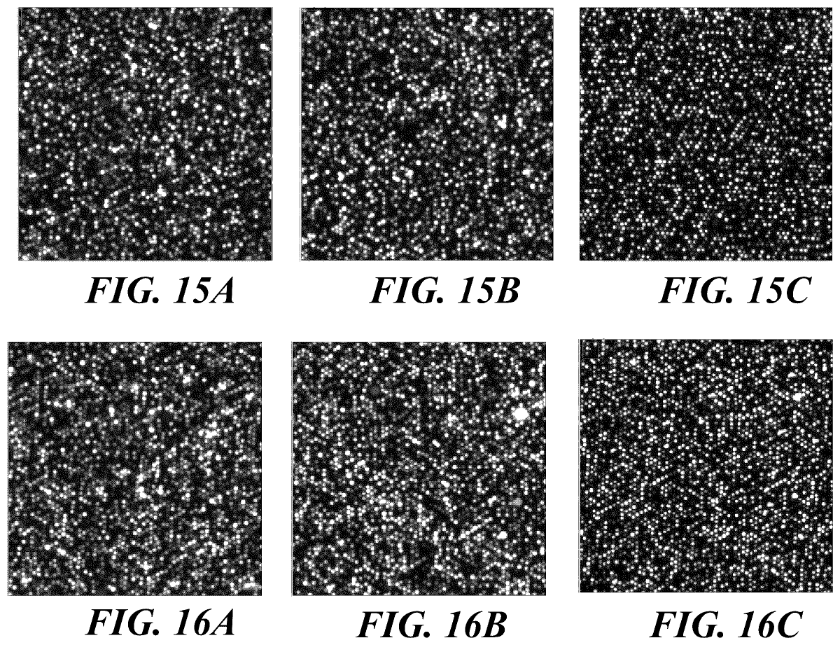

The sizes of the beads in the presence of a shrinking buffer (i.e., 20 mM Tris, 80 mM NaCl, 0.05% w/v Triton X-100, 10% w/v PEG-4000, and 50 mM Mg²⁺—FIG. 15C) were smaller than those in the presence of a control imaging buffer (i.e., 20 mM Tris, 80 mM NaCl, and 0.05% w/v Triton X-100—FIG. 15A) or than those in the presence of a shrinking buffer without PEG (i.e., 20 mM Tris, 80 mM NaCl, and TritonX0.05% w/v Triton X-100 and 50 mM Mg²⁺—FIG. 15B). Similar shrinkage of the beads was also observed in the presence of PEG-8000 (e.g., in a shrinking buffer comprising 20 mM Tris, 80 mM NaCl, 0.05% w/v Triton X-100, 10% w/v PEG-8000, and 50 mM Mg²⁺). The sizes of the beads in the image taken in the presence of a shrinking buffer comprising PEG-8000 (see FIG. 16C) were smaller than those taken in the presence of the control imaging buffer (20 mM Tris, 80 mM NaCl, TritonX) (see FIG. 16A) or the shrinking buffer without PEG (see FIG. 16B). The Mg²⁺ present in the different shrinking buffers was derived from MgCl₂.

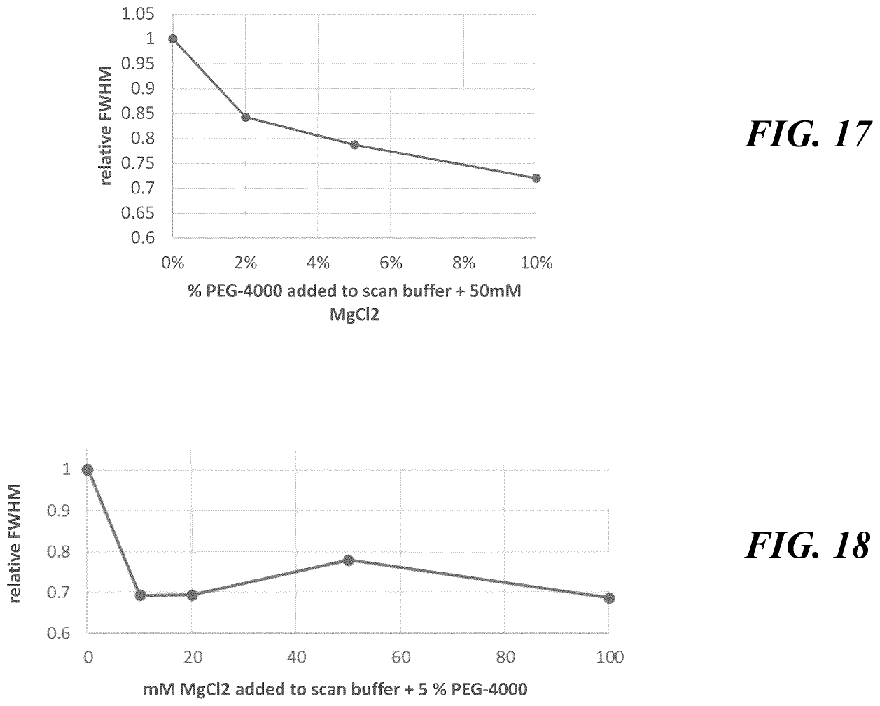

The average FWHM of beads in the first test imaging buffer (20 mM Tris, 80 mM NaCl, TritonX) was measured at 1.11 um. In contrast, the average FWHM of beads in the presence of the fifth imaging buffer (20 mM Tris, 80 mM NaCl, TritonX, 10% w/v PEG-8000, 50 mM Mg²⁺) was measured at 0.82 μm, about 30% (1.11 μm) smaller than that imaged under the control imaging buffer. The accumulation of this data suggests that both polymers (e.g., PEG) and cations (e.g., Mg²⁺) can decrease the size of the bead when added in the imaging buffer. For example, as shown in FIG. 17, increasing the amount of PEG in the shrinkage imaging buffer (20 mM Tris, 80 mM NaCl, TritonX, 50 mM Mg²⁺) decreased the average size of the beads. 10% PEG could decrease the average bead size by greater than 25%. Furthermore, as shown in FIG. 18, concentrations as low as 10 mM Mg²⁺ added to the shrinkage imaging buffer (20 mM Tris, 80 mM NaCl, TritonX, 10% w/v PEG). Thus, the addition of a combination of PEG and Mg²⁺ has been demonstrated to decrease average bead size after beads are loaded onto substrates.

### Example 4: Loading Beads with Double-Stranded DNA to Generate Monolayers of Beads on Unpatterned Substrates

Sequencing beads may be loaded onto a substrate already coupled to nucleic acid molecules (e.g., templates to be sequenced or primer sequences to capture templates). Single stranded DNA has the capacity and tendency to anneal to other strands of single stranded DNA (e.g., through both canonical and non-canonical base-pairing). Beads for sequencing typically comprise (e.g., each bead is coupled to) hundreds or thousands or tens of thousands of strands of DNA. Where these DNA molecules are single-stranded, they can anneal to themselves and to other single stranded template molecules (e.g., on the same bead or on adjacent beads) and cause beads to associate closely with each other both in solution and on the substrate. Clumps (e.g., aggregations of beads) make it difficult to identify the sequences of individual template molecules or colonies of identical template molecules (e.g., from fluorescent-based nucleotide base incorporations). As seen in Example 1, beads coupled to single-stranded nucleic acid molecules are capable of forming monolayers (e.g., self-assembling) but do show some propensity for aggregation. The effect of loading beads coupled to double-stranded nucleic acid molecules on bead self-assembly is interrogated.

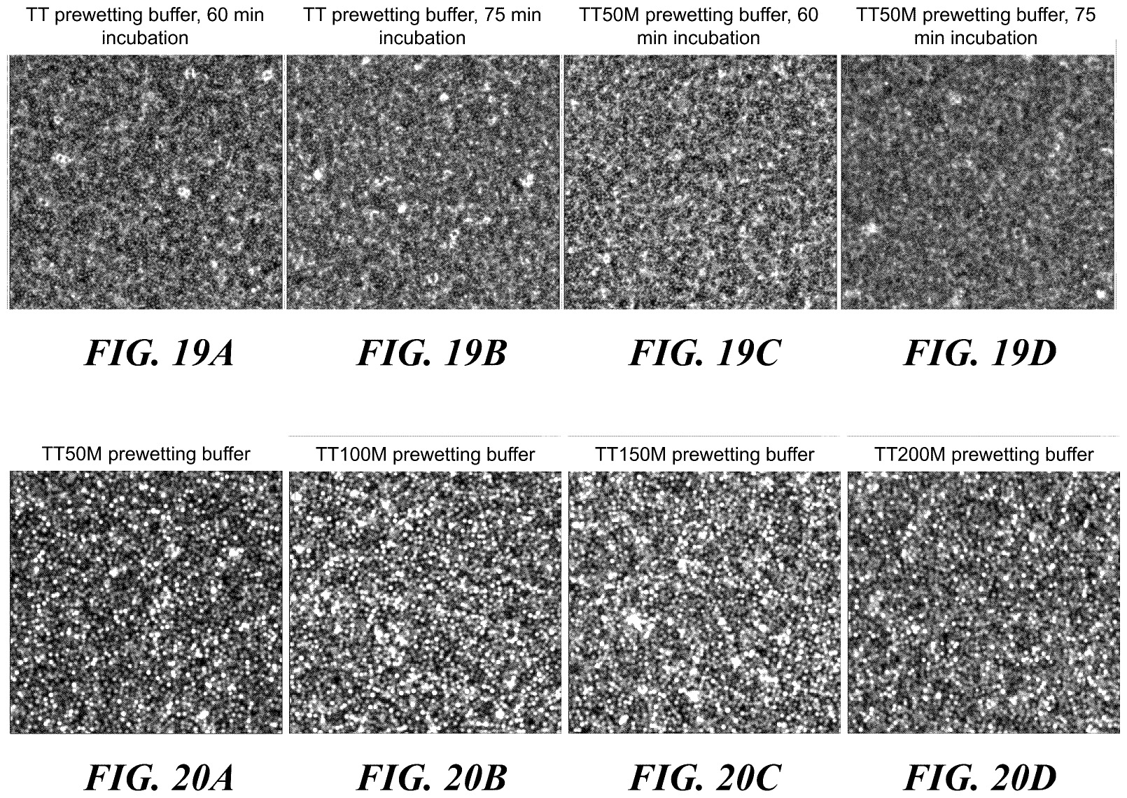

Experimental Conditions: To interrogate the impact of inter-bead nucleic acid interactions on loading and bead self-assembly, the following experiments were performed with sequencing beads coupled to double-stranded nucleic acid molecules (e.g., dsDNA beads). In each experiment, the dsDNA beads were prepared in “TT” loading buffer (e.g., 10 mM Tris pH7 and 0.05% Tergitol), unless noted otherwise. As described in Example 2, an unpatterned substrate (2 cm×2 cm coupons of substrate) was prepared, prior to loading in prewetting “TT50M” buffer (10 mM Tris pH7, 0.05% Tergitol, and 50 mM MgCl₂), unless noted otherwise. 15 μL of beads (or 16.7 uL for FIGS. 19A-19D) were dispensed onto the substrate via drop loading, as described in Example 1. The bead-loaded substrate was then incubated at room temperature with rotation at about 2 rpm, for about 60 minutes, unless noted otherwise. After incubation (during which the loaded beads formed a self-assembled monolayer adjacent to the substrate), the bead-loaded substrate was washed with wash buffer (“WB24”) comprising 20 mM Tris pH 8.8, 0.08M NaCl, and 0.1% Triton X-100. Occupancy calculations were performed based on a predetermined pitch (e.g., center-to-center average bead distance). Representative images of beads (e.g., fluorescence from labeled bead or template primers) dispersed along the substrate, for each condition described below, were obtained using a 20x microscope objective.

Effect of drying time and substrate prewetting buffer: FIGS. 19A-19D illustrate beads loaded onto substrates for self-assembly when the incubation time and the prewetting buffer are modified. The beads were amplified with 300 bp template molecules. In FIGS. 19A-19B the prewetting buffer was “TT”, and in FIGS. 19C-19D the prewetting buffer was “TT50M”. In FIGS. 19A and 19C the incubation time was 60 minutes, and in FIGS. 19B and 19D the incubation time was 75 minutes. In each case, 16.7 uL of beads were dispensed onto the substrates, and the concentration of beads was 30 million/uL. The predetermined pitch used for bead occupancy calculations was 1.2 μm. For all conditions, 68.6% of the loaded beads were template-positive (e.g., beads that were coupled to nucleic acid template molecules). Substrate occupancy was determined based on the total number of beads loaded.

TABLE 4
Substrate bead occupancy resulting from altering incubation
time and prewetting buffer, with a pitch of 1.2 μm.

| | | | | | Derived | |
|---|---|---|---|---|---|---|
| | Prewetting | Incubation | Average | Occupancy | pitch | FWHM |
| FIG. | buffer | time | occupancy | STD | (average) | (average) |
| FIG. 19A | TT | 60 minutes | 77% | 9% | 1.16 μm | 0.888 μm |
| FIG. 19B | TT | 75 minutes | 79% | 8% | 1.13 μm | 0.932 μm |
| FIG. 19C | TT50M | 60 minutes | 82% | 2% | 1.18 μm | 0.945 μm |
| FIG. 19D | TT50M | 75 minutes | 81% | 3% | 1.18 μm | 0.921 μm |

In each experiment, the derived pitch (e.g., the average center-to-center distance between adjacent beads) was similar. As seen in Table 4, the average occupancy was higher when the prewetting buffer was TT50M (10 mM Tris pH7, 0.05% Tergitol, and 50 mM MgCl₂). There did not appear to be much difference when the incubation time was increased from 60 to 75 minutes. Average FWHM (full-width half-maximum) was determined from grey-level fluorescence intensities of beads in the images, and this can be interpreted as the average diameter of the loaded beads.

Effect of substrate prewetting buffer: FIGS. 20A-20D illustrate bead loading and self-assembly on substrates that were prepared with different prewetting buffers. Beads were amplified with 500 bp template molecules. For FIGS. 20A-20D, the prewetting TT buffers had 50 mM MgCl₂, 100 mM MgCl₂, 150 mM MgCl₂, or 200 mM MgCl₂, respectively. In each case, the incubation time was 60 minutes. In each case, the average occupancy was determined based on a 1.4 μm pitch. 76% of the loaded beads were template-positive, and substrate occupancy was determined based on template-positive beads.

TABLE 5
Substrate bead occupancy from altering prewetting buffer,
with a pitch of 1.4 μm.

| | | | | Derived | |
|---|---|---|---|---|---|
| | Prewetting | Average | Occupancy | pitch | FWHM |
| FIG. | buffer | occupancy | STD | (Average) | (average) |
| FIG. 20A | TT50M | 76% | 2% | 1.46 μm | 1.24 μm |
| FIG. 20B | TT100M | 77% | 1% | 1.45 μm | 1.08 μm |
| FIG. 20C | TT150M | 78% | 0% | 1.42 μm | 1.16 μm |
| FIG. 20D | TT200M | 77% | 0% | 1.44 μm | 1.12 μm |

In agreement with the results in Example 2, the exact amount of magnesium chloride (i.e., magnesium ions) had little impact on average occupancy here (see Table 5).

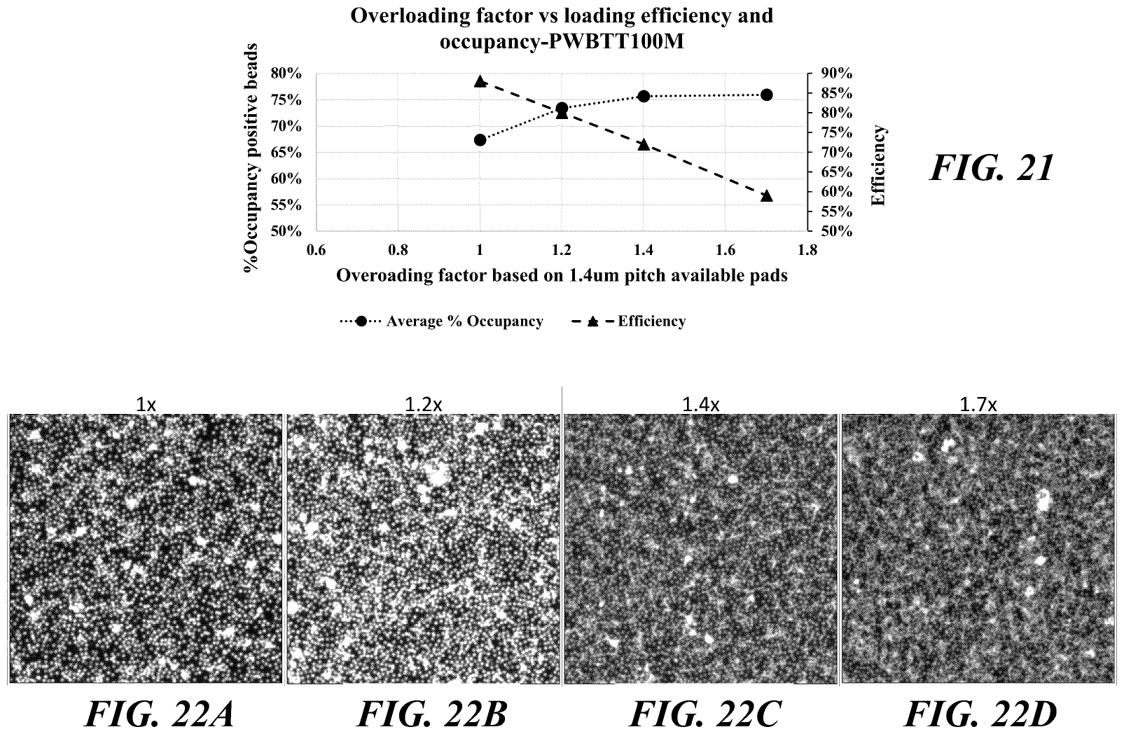

Impact of bead overloading on occupancy: FIGS. 21-22D illustrate the impact of the total number of beads in the plurality of beads on loading efficacy in self-assembly. Beads were amplified with 500 bp template molecules. With a patterned substrate, there are a known number of available locations for beads/particles to adhere to. However, in an unpatterned or at least partially unpatterned substrate (e.g., suitable for self-assembly) the number of available individually addressable locations depends on bead/particle size and packing efficiency and the number of loaded beads/particles. Overloading is closely related to pitch (e.g., derived or possible pitch), which in turn depends on the overall size of the beads/particles being loaded (e.g., the unamplified size of beads and the length of template molecules amplified on the beads).

FIG. 21 tracks both the average occupancy (e.g., the percentage of projected/possible individually addressable locations that are occupied) of template-positive beads and the loading efficiency (e.g., the percentage of loaded beads that ultimately remain on the substrate) with respect to the total number of beads loaded, as illustrated in FIGS. 22A-22D. The substrate in each case was prewet with a TT buffer comprising 100 mM MgCl₂. In each case, of the beads that were dispensed onto the substrate, 76% were template-positive. The amount of overloading was determined from the assumed pitch of 1.4 μm. That is, a total bead overloading factor was determined by estimating a total number of individually addressable locations using the pitch 1.4 μm.

FIG. 21 shows a clear decrease in loading efficiency as the overloading factor increased (detailed also in Table 6). However, the average occupancy plateaued at about 75%, despite the increase in overloading factor. In this case, average occupancy was determined for template-positive beads. There is a tradeoff in loading beads onto a substrate for self-assembly in that as more beads are dispensed, the possible density of beads on the substrate increases (thus increasing overall sequencing efficiency), however, as more beads are dispensed, there is also an increased likelihood of bead clumping/aggregation (which decreases sequencing efficacy). There is thus a strong interest in determining an optimum number of beads to load. As seen in FIGS. 22A-22D, there was some increase in aggregation corresponding to the degrees of overloading tested. Given the tension between aggregation and efficient use of substrate space, in some cases it may be preferrable to ‘overload’ a substrate.

TABLE 6
Substrate bead occupancy resulting from bead overloading, with a pitch of 1.4 μm.

| | | Total | Positive | | | | | |
|---|---|---|---|---|---|---|---|---|
| | Total # | Bead over- | bead over- | | | Derived | | |
| | dispensed | loading | loading | Average | Occupancy | pitch | Loading | FWHM |
| FIG. | beads | factor | factor | occupancy | STD | (average) | efficiency | (average) |
| FIG. | 236 | 1 | 0.76 | 67% | 2% | 1.56 μm | 88% | 1.19 μm |
| 22A | million | | | | | | | |
| FIG. | 282 | 1.2 | 0.91 | 73% | 2% | 1.48 μm | 80% | 1.22 μm |
| 22B | million | | | | | | | |
| FIG. | 330 | 1.4 | 1.06 | 76% | 1% | 1.52 μm | 72% | 1.14 μm |
| 22C | million | | | | | | | |
| FIG. | 405 | 1.7 | 1.3 | 76% | 1% | 1.49 μm | 59% | 1.15 μm |
| 22D | million | | | | | | | |

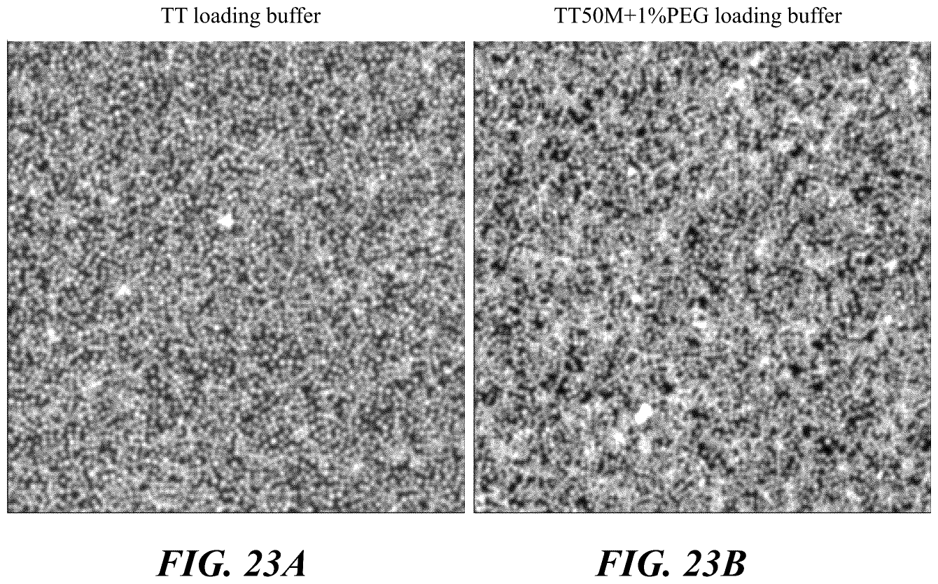

Impact of a shrinking agent: FIGS. 23A and 23B show examples of bead self-assembly with modifications to the loading buffer. The beads were amplified with 300 bp template molecules. In FIG. 23A, the loading buffer lacked magnesium chloride and PEG. In FIG. 23B, the loading buffer contained 50 mM MgCl₂ and 10 w/v of PEG-4000. The addition of PEG and magnesium chloride, as described elsewhere herein, As can be seen in the figures and from Table 7, there was no substantial difference in average occupancy or other bead factors based on these loading buffer modifications. In some cases, PEG can act as a shrinking agent (see e.g., Example 3) and serve to decrease the size of beads (e.g., by removing water from hydrogel beads). In some cases, this can lead to improvements in loading occupancy. This was not observed here; however, in some cases, higher amounts of PEG may increase bead occupancy (not shown). Bead occupancy was determined based on total beads loaded.

TABLE 7
Substrate bead occupancy resulting from altering
loading buffer, with a pitch of 1.2 μm.

| | Loading | Loading | | | Derived | |
|---|---|---|---|---|---|---|
| | buffer | buffer | Average | Occupancy | pitch | FWHM |
| FIG. | MgCl₂ | PEG-4000 | occupancy | STD | (Average) | (average) |
| FIG. 23A | 0 mM | 0% w/v | 84% | 1% | 1.17 μm | 0.922 μm |
| FIG. 23B | 50 mM | 1% w/v | 82% | 1% | 1.16 μm | 1.00 μm |

### Example 5: Bead Self-Assembly for Amplification

In some cases, it may be advantageous to perform amplification of template nucleic acid molecules on a substrate (e.g., not in solution prior to loading onto the substrate, as in Example 4). Such amplification methods may generate concatemers. In some cases, amplification may be performed in a single stage. In some cases, amplification may be performed in multiples stages, such as two stages or more. Amplification may comprise rolling circle amplification (RCA) and/or multiple displacement amplification (MDA). Such devices, systems, methods, compositions, and kits can be applied alternatively or in addition to the various operations described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

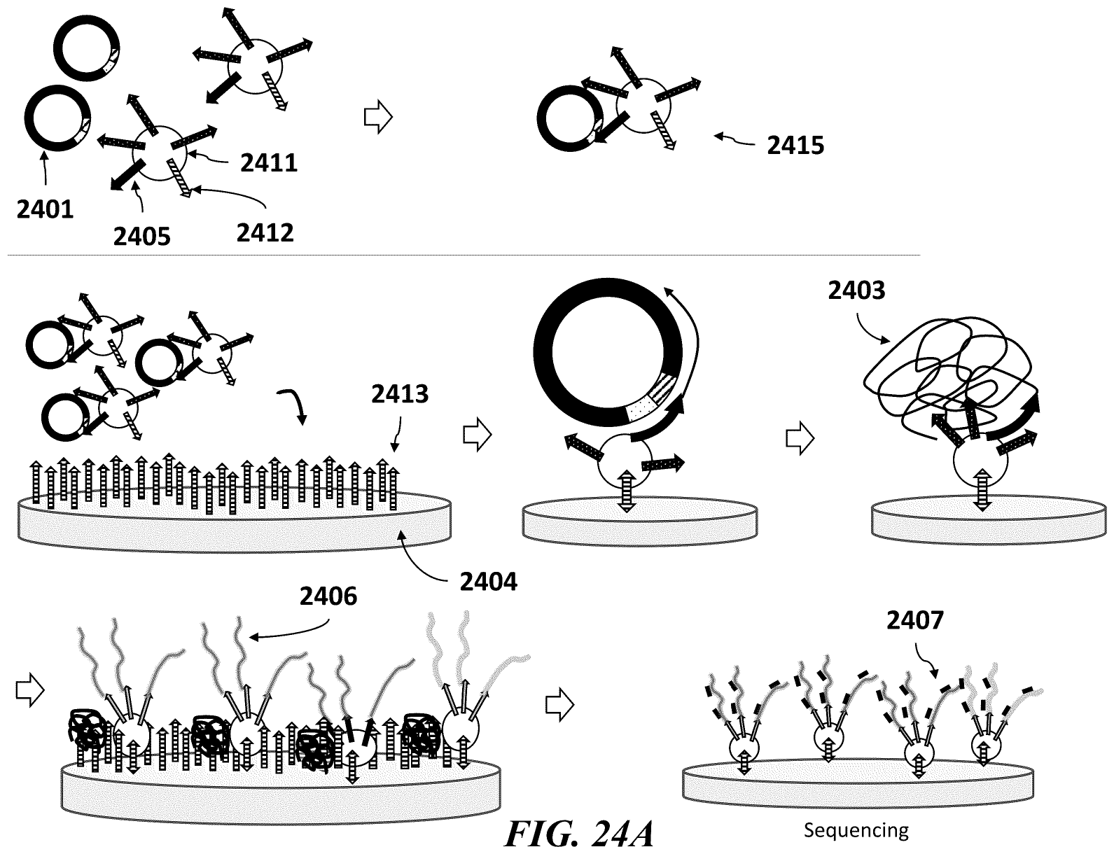

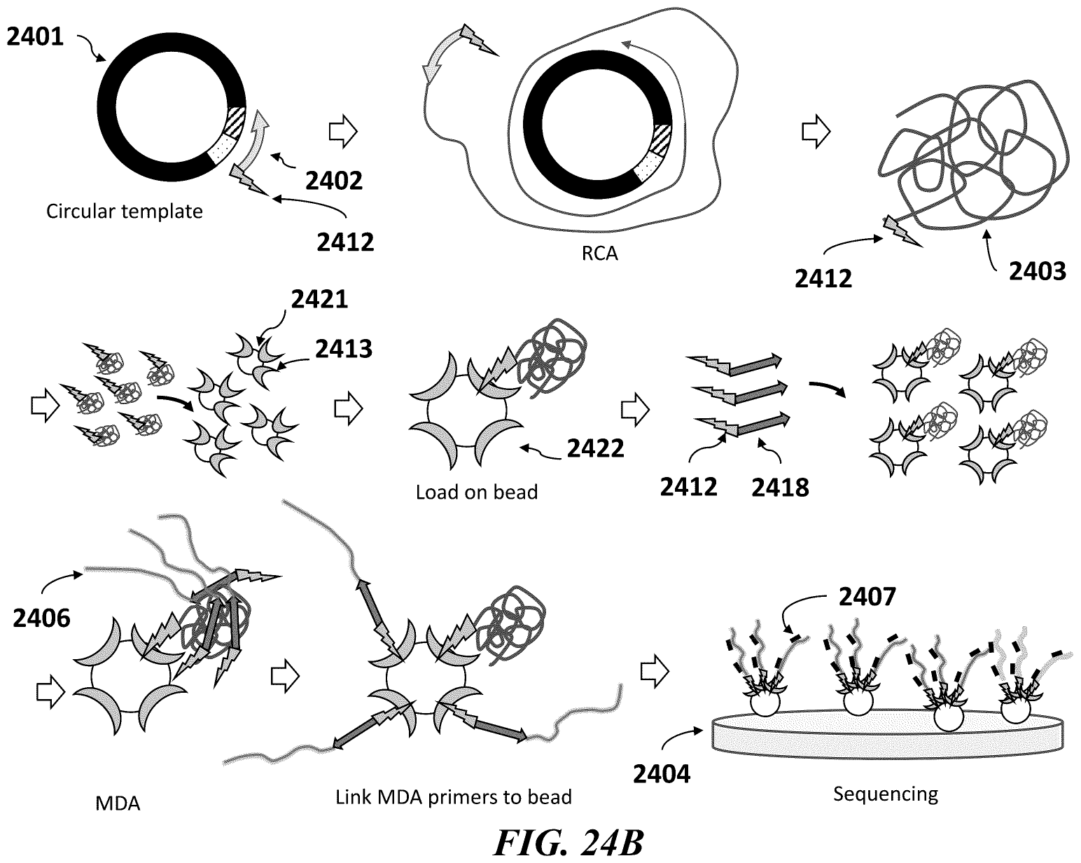

A template nucleic acid molecule may be circularized prior to amplification. A circular template (e.g., 2401 in FIGS. 24A and 24B) may comprise a first adapter, a template nucleic acid molecule, and a second adapter. A concatemer may comprise at least two repeating oligonucleotide units. In an example, an oligonucleotide unit comprises a first adapter, a template nucleic acid molecule, and a second adapter, in the listed order or other orders.

In FIG. 24A, circular templates (e.g., 2401) may be bound to beads 2411 to form a bead assembly 2415 (bead-bound circular template). A bead may comprise a plurality of surface primers 2405 and click chemistry couplers 2412. One of the surface primers of the bead may bind to the circular template 2401 at the first adapter and/or second adapter to form the bead assembly. The bead assemblies may be deposited (e.g., loaded for self-assembly) onto a substrate 2404 coated with complementary click chemistry couplers 2413. The click chemistry couplers 2412 of the beads 2411 may be configured to couple with the complementary click chemistry couplers 2413 via click chemistry pairings. After deposition, in some cases, the bead assemblies may be spaced apart from each other via the beads (e.g., 2411) acting as spacers. In some cases, the beads may self-assemble themselves as a layer on the substrate 2404. Effectively, each location of a bead assembly may become an individually addressable location. The bead assemblies (e.g., 2415) may be immobilized to the substrate 2404 by coupling the couplers 2412 and 2413. The circular templates may be amplified on the substrate using the surface primers 2405 on the bead (e.g., 2411) such as via RCA and/or MDA to generate concatemers in the forward (e.g., 2403) and reverse (e.g., 2406) directions. It will be appreciated that surface primers 2405 include both forward and reverse primers. After amplification, a plurality of concatemers may be immobilized to the substrate via the beads. One type of concatemer (forward or reverse, separately) may then be sequenced on the substrate according to systems and methods described herein, such as by providing a plurality of sequencing primers 2407. The sequencing signals collected from each individually addressable location producing a signal may be attributed to a single distinct template insert. In some cases, the other type of concatemer may additionally be sequenced.

A click chemistry coupler and complementary click chemistry coupler pair may comprise functional groups configured to form covalent bonds upon reaction by click chemistry (e.g., Staudinger ligation or Diels-Alder chemistry) or by a click reaction. Coupling pairs are well known in the art. Examples of coupling pairs include, but are not limited to, biotin-avidin, carboxylic acid-amino group, NHS ester-amino group, maleimide-thiol, and Azide-DBCO.

In FIG. 24B, in a first stage amplification, a circular template 2401 may be amplified in solution using a solution primer 2402 coupled to a click chemistry coupler 2412, such as via RCA to generate a first stage concatemer 2403 coupled to the click chemistry coupler 2412. The solution primer 2402 may bind to the circular template 2401 at the first adapter and/or second adapter. The first stage concatemer 2403 and other first stage concatemers generated from the template library may be coupled to coupling beads 2421 to generate bead assemblies 2422 (bead-bound first stage concatemers). Each coupling bead may be coated with complementary click chemistry couplers 2413 which can react with the click chemistry couplers 2412 on the first stage concatemers 2403. A plurality of primers 2418 each coupled to a click chemistry coupler 2412 may be provided to the bead assemblies 2422. A single first stage concatemer may be bound to multiple primers of the plurality of primers 2418 at the bead. In a second stage amplification, the first stage concatemers (e.g., 2403) bound to the beads (e.g., 2421) may be amplified on the beads using the primers 2418, such as via MDA to generate second stage concatemers 2406. It will be appreciated that the primers 2418 can include forward and reverse primers, and during second stage amplification, concatemers in the forward and reverse directions can be generated, such as via RCA and/or MDA. The primers 2418 may be coupled to the beads (e.g., 2421) via the couplers 2412, 2413 prior to, during, or subsequent to the second stage amplification. A plurality of concatemers may be immobilized to the beads. The bead-bound second stage concatemers may be deposited onto a substrate 2404. The substrate may or may not be patterned with individually addressable locations, the individually addressable locations discretely spaced apart from each other. In some cases, where the substrate is not patterned, the bead-bound second stage concatemers may be spaced apart from each other via the beads (e.g., 2421) acting as spacers. In some cases, the beads may self-assemble themselves as a layer on the substrate 2404. Effectively, each location of a bead-bound second stage concatemer may become an individually addressable location on the substrate. After deposition, at most one bead-bound second stage concatemer can be immobilized to each individually addressable location. The second stage concatemers 2406 may then be sequenced on the substrate according to systems and methods described herein, such as by providing a plurality of sequencing primers 2407. The sequencing primers may be hybridized to second stage concatemers 2406 on or off the substrate 2404. The sequencing signals collected from each individually addressable location producing a signal may be attributed to a single distinct template insert.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A method for self-assembly of a plurality of beads, comprising: (a) providing a substrate and said plurality of beads, wherein said substrate is unpatterned and substantially planar; (b) dispensing said plurality of beads adjacent to said substrate; and (c) subjecting said substrate or said plurality of beads to conditions sufficient for self-assembly of said plurality of beads adjacent to said substrate.

2. A method for self-assembly of a plurality of beads, comprising: (a) providing a substrate and said plurality of beads, wherein said substrate is at least partially unpatterned and substantially planar; (b) dispensing said plurality of beads adjacent to said substrate; and (c) subjecting said substrate or said plurality of beads to conditions sufficient for self-assembly of said plurality of beads adjacent to said substrate.

3. A method for self-assembly of a plurality of beads, comprising: (a) providing a substrate and said plurality of beads, wherein said substrate is nanopatterned and substantially planar; (b) dispensing said plurality of beads adjacent to said substrate; and (c) subjecting said substrate or said plurality of beads to conditions sufficient for self-assembly of said plurality of beads adjacent to said substrate.

4. A method for self-assembly of a plurality of beads, comprising: (a) dispensing the plurality of beads onto a substrate for self-assembly, wherein the substrate is unpatterned and substantially planar; and (b) sequencing a plurality of amplified products immobilized to the plurality of beads. 5. The method of embodiment 4, further comprising: prior to the dispensing (a), amplifying a plurality of analytes coupled to the plurality of beads to produce the plurality of amplified products immobilized to the plurality of beads. 6. The method of embodiment 4, further comprising: after to the dispensing (a) and prior to the sequencing (b), amplifying a plurality of analytes coupled to the plurality of beads to produce the plurality of amplified products immobilized to the plurality of beads.

7. The method of embodiment 2, wherein a first portion of the substrate is unpatterned and a second portion of the substrate is patterned. 8. The method of embodiment 3, wherein the nanopatterned substrate comprises two or more topological features with a diameter less than 50 nm. 9. The method of embodiment 8, wherein each bead in said plurality of beads contacts at least two features.

10. The method of any one of embodiments 1-4, wherein said substrate is a solid or semi-solid substrate. 11. The method of any one of embodiments 1-4, wherein said plurality of beads is a plurality of solid or semi-solid beads. 12. The method of any one of embodiments 1-4, wherein said plurality of beads is formed from a metal, a ceramic, a polymer, or glass, or a combination thereof. 13. The method of embodiment 12, wherein said polymer is a gel. 14. The method of embodiment 13, wherein said gel is a hydrogel.

15. The method of any one of embodiments 1-4, wherein said plurality of beads is electrostatically charged. 16. The method of embodiment 15, wherein said plurality of beads is negatively charged, and wherein said substrate or portion thereof is positively charged. 17. The method of embodiment 16, wherein said portion thereof is a surface of said substrate.

18. The method of any one of embodiments 1-4, wherein said plurality of beads comprises a plurality of nucleic acid molecules coupled thereto. 19. The method of any one of embodiments 1-4, wherein said plurality of beads comprises a first subset of beads comprising a plurality of nucleic acid molecules coupled thereto, and a second subset of beads that do not comprise a plurality of nucleic acid molecules coupled thereto. 20. The method of embodiment 18 or 19, wherein said plurality of nucleic acid molecules comprises a deoxyribonucleic acid (DNA) molecule. 21. The method of embodiment 20, wherein said DNA molecule is double-stranded. 22. The method of embodiment 20, wherein said DNA molecule is single-stranded. 23. The method of any one of embodiments 18-22, wherein the plurality of nucleic acid molecules comprises a plurality of nucleic acid nanoballs. 24. The method of embodiment 22, wherein, in (a), said plurality of beads is provided in a solution, wherein said solution comprises single-stranded binding proteins.

25. The method of embodiment 20, further comprising, sequencing said plurality of nucleic acid molecules. 26. The method of embodiment 25, wherein said sequencing comprises flow sequencing, which flow sequencing comprises (i) providing a reagent comprising a first plurality of nucleotides to said plurality of beads or said substrate and (ii) detecting a nucleotide from said first plurality of nucleotides. 27. The method of embodiment 26, wherein said flow sequencing further comprises (iii) providing an additional reagent comprising a second plurality of nucleotides to said plurality of beads or said substrate and (iv) detecting an additional nucleotide from said second plurality of nucleotides. 28. The method of embodiment 27, wherein said first plurality and said second plurality of nucleotides are of a same nucleotide base type. 29. The method of embodiment 27, wherein said first plurality and said second plurality of nucleotides are of different nucleotide base types.

30. The method of any one of embodiments 1-4, wherein beads of said plurality of beads are from about 0.1 microns to about 10 microns in diameter. 31. The method of embodiment 30, wherein at least a first bead in said plurality of beads has a first diameter and a second bead in said plurality of beads has a second diameter.

32. The method of any one of embodiments 1-3, further comprising, prior to (b), wetting said substrate. 33. The method of embodiment 32, wherein said wetting comprises wetting said substrate with an ionic buffer. 34. The method of embodiment 33, wherein said ionic buffer comprises magnesium. 35. The method of embodiment 34, wherein said ionic buffer comprises magnesium chloride. 36. The method of embodiment 35, wherein said magnesium chloride is provided at a molarity of from about 10 to about 50 millimolar (mM). 37. The method of embodiment 32, wherein said wetting renders said substrate hydrophilic. 38. The method of any one of embodiments 1-3, further comprising, treating said substrate prior to (b). 39. The method of embodiment 38, wherein said treating comprises depositing a silane adjacent to said substrate. 40. The method of embodiment 39, wherein said silane is an amino silane. 41. The method of embodiment 40, wherein said amino silane is 3-aminopropyltrimethoxysilane (APTMS). 42. The method of embodiment 39, wherein said silane is deposited on said substrate using vapor deposition.

43. The method of embodiment 42, wherein said substrate comprises a silicon wafer. 44. The method of embodiment 42, wherein said substrate comprises a silicon oxide layer. 45. The method of embodiment 42, wherein said substrate comprises a glass wafer. 46. The method of embodiment 45, wherein said glass wafer comprises a liquid crystal display. 47. The method of embodiment 1 or 4, wherein said substrate does not comprise topographical features.

48. The method of any one of embodiments 1-3, wherein (a) comprises providing said plurality of beads in a solution, and wherein (b) comprises contacting said substrate with said solution. 49. The method of embodiment 48, wherein (b) comprises providing said solution in one or more droplets to said substrate and allowing said solution to spread adjacent to said substrate. 50. The method of embodiment 49, wherein said allowing comprises incubating said solution on said substrate. 51. The method of embodiment 50, wherein said incubating is performed for about 20 to about 120 minutes.

52. The method of embodiment 48, further comprising, rotating said substrate to disperse said plurality of beads across said substrate. 53. The method of embodiment 52, wherein said rotating is performed from about 1 revolution per minute (rpm) to about 8000 revolutions per minute (rpm). 54. The method of embodiment 52, further comprising drying said substrate subsequent to said rotating. 55. The method of embodiment 54, wherein said drying is performed for about 20 minutes. 56. The method of embodiment 54, wherein said drying is performed at ambient temperature. 57. The method of embodiment 54, wherein said drying is performed at a temperature from about 20 degrees Celsius to about 200 degrees Celsius.

58. The method of embodiment 48, wherein (b) comprises translating said substrate relative to said solution, thereby contacting said substrate with said plurality of beads. 59. The method of embodiment 58, wherein said contacting is performed for from about 1 minute to about 60 minutes. 60. The method of embodiment 59, wherein said contacting is performed for about 60 minutes. 61. The method of embodiment 59, wherein said contacting is performed for about 120 minutes. 62. The method of embodiment 48, further comprising, drying said substrate subsequent to said contacting. 63. The method of embodiment 62, wherein said drying is performed for from about 1 minute to about 60 minutes. 64. The method of embodiment 63, wherein said drying is performed for 15 minutes. 65. The method of embodiment 63, wherein said drying is performed for 30 minutes.

66. The method of any one of embodiments 1-3, wherein, subsequent to (c), said plurality of beads is arranged in a self-assembled monolayer. 67. The method of embodiment 66, wherein said self-assembled monolayer is substantially uniform. 68. The method of embodiment 67, wherein said self-assembled monolayer has a bead-to-bead tolerance of approximately twice the diameter of a bead of said plurality of beads. 69. The method of embodiment 66, wherein said self-assembled monolayer is arranged such that individual beads of said plurality of beads are distinguishable by microscopy.

70. The method of any one of embodiments 1-4, further comprising, subjecting said substrate to conditions sufficient to decrease the size of said plurality of beads. 71. The method of embodiment 70, wherein said conditions sufficient to decrease the size of said plurality of beads comprises applying a shrinking buffer to said plurality of beads. 72. The method of embodiment 71, wherein said shrinking buffer comprises polyethylene glycol (PEG). 73. The method of embodiment 72, wherein said PEG comprises PEG 4000 or PEG 8000. 74. The method of embodiment 72, wherein said PEG is provided at a 10% w/v concentration.

75. The method of embodiment 71, wherein said shrinking buffer comprises magnesium salts. 76. The method of embodiment 75, wherein said magnesium salts comprise magnesium chloride. 77. The method of embodiment 76, wherein said magnesium chloride is provided at a 50 mM concentration. 78. The method of embodiment 71, wherein said shrinking buffer comprises spermine. 79. The method of embodiment 78, wherein said spermine is provided at a molarity from about 5 mM to about 50 mM.

80. A kit, comprising: a substrate, wherein said substrate is unpatterned and substantially planar; a plurality of beads; and instructions for forming a self-assembled monolayer of said plurality of beads adjacent to said substrate. 81. The kit of embodiment 80, wherein said substrate is a solid or semi-solid substrate. 82. The kit of embodiment 80, wherein said plurality of beads is a plurality of solid or semi-solid beads. 83. The kit of embodiment 80, wherein said plurality of beads comprises a plurality of nucleic acid molecules coupled thereto. 84. The kit of embodiment 83, wherein said plurality of nucleic acid molecules comprises a deoxyribonucleic acid (DNA) molecule. 85. The kit of embodiment 84, wherein said DNA molecule is double-stranded. 86. The kit of embodiment 80, further comprising, a pre-wetting buffer. 87. The kit of embodiment 86, wherein said pre-wetting buffer comprises an ionic buffer. 88. The kit of embodiment 87, wherein said ionic buffer comprises magnesium. 89. The kit of embodiment 80, further comprising, a shrinking buffer. 90. The kit of embodiment 89, wherein said shrinking buffer comprises polyethylene glycol (PEG). 91. The kit of embodiment 89, wherein said shrinking buffer comprises magnesium salts. 92. The kit of embodiment 91, wherein said magnesium salts comprise magnesium chloride.

93. A system, comprising: a substrate, wherein said substrate is unpatterned and substantially planar; and a plurality of beads, wherein: at least a first subset of said plurality of beads is in a substantially close-packed configuration, and at least a second subset of said plurality of beads is in a substantially monolayer configuration. 94, The system of embodiment 93, wherein said substrate is a solid or semi-solid substrate. 95. The system of embodiment 93, wherein said plurality of beads is a plurality of solid or semi-solid beads. 96. The system of embodiment 95, wherein said close-packed configuration comprises a center-to-center distance between neighboring beads of from 1 μm to 1.8 μm. 97. The system of embodiment 93, wherein said plurality of beads comprises a plurality of nucleic acid molecules coupled thereto. 98. The system of embodiment 97, wherein said plurality of nucleic acid molecules comprises a deoxyribonucleic acid (DNA) molecule. 99. The system of embodiment 98, wherein said DNA molecule is double-stranded. 100. The system of embodiment 93, wherein said first subset and said second subset are the same or substantially the same.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-51. (canceled)

52. A method for self-assembly of a plurality of beads, comprising:
(a) providing a substrate and said plurality of beads, wherein said substrate is unpatterned and substantially planar;
(b) dispensing said plurality of beads adjacent to said substrate; and
(c) subjecting said substrate or said plurality of beads to conditions sufficient for self-assembly of said plurality of beads adjacent to said substrate.

53. The method of claim 52, wherein said substrate is a solid or semi-solid substrate, and wherein said plurality of beads is a plurality of solid or semi-solid beads.

54. The method of claim 52, wherein said plurality of beads comprises a plurality of nucleic acid molecules coupled thereto.

55. The method of claim 54, further comprising, sequencing said plurality of nucleic acid molecules.

56. The method of claim 55, wherein said sequencing comprises flow sequencing, which flow sequencing comprises (i) providing a reagent comprising a first plurality of nucleotides to said plurality of beads or said substrate, (ii) detecting a nucleotide from said first plurality of nucleotides, (iii) providing an additional reagent comprising a second plurality of nucleotides to said plurality of beads or said substrate, and (iv) detecting an additional nucleotide from said second plurality of nucleotides.

57. The method of claim 52, wherein beads of said plurality of beads are from about 0.1 microns to about 10 microns in diameter.

58. The method of claim 52, further comprising, prior to (b), wetting said substrate.

59. The method of claim 58, wherein said wetting comprises wetting said substrate with an ionic buffer.

60. The method of claim 59, wherein said ionic buffer comprises magnesium.

61. The method of claim 58, wherein said wetting renders said substrate hydrophilic.

62. The method of claim 52, wherein said substrate comprises a silicon wafer.

63. The method of claim 52, wherein said substrate comprises a silicon oxide layer.

64. The method of claim 52, wherein said substrate comprises a glass wafer.

65. The method of claim 52, wherein (a) comprises providing said plurality of beads in a solution, and wherein (b) comprises contacting said substrate with said solution.

66. The method of claim 65, wherein (b) comprises providing said solution in one or more droplets to said substrate and allowing said solution to spread adjacent to said substrate.

67. The method of claim 65, further comprising, rotating said substrate to disperse said plurality of beads across said substrate.

68. The method of claim 67, wherein said rotating is performed from about 1 revolution per minute (rpm) to about 8000 rpm.

69. The method of claim 52, wherein, subsequent to (c), said plurality of beads is arranged in a self-assembled monolayer.

70. The method of claim 69, wherein said self-assembled monolayer has a bead-to-bead tolerance of approximately twice a diameter of a bead of said plurality of beads.

71. The method of claim 69, wherein said self-assembled monolayer is arranged such that individual beads of said plurality of beads are distinguishable by microscopy.
